(19) United States
(12) Patent Application Publication
(10) Pub. No.: US 2003/0152925 A1
(43) Pub. Date: Aug. 14, 2003

(54) Annealing control primer system for regulating primer annealing specificity and its applications

(76) Inventor: Jong-Yoon Chun, Seoul (KR)

Correspondence Address: Judith L. Toffenetti, Esq., McDermott, Will & Emery, 600 13th Street, N.W., Washington, DC, 20005-3096, US

(21) Appl. No.: 10/014,496

(22) Filed: Dec. 14, 2001

(30) Foreign Application Priority Data
Dec. 8, 2001 PCT/KR01/02133

(51) Int. Cl. C12Q 1/68; C07H 21/04

(52) U.S. Cl. 435/6; 536/24.3

(57) ABSTRACT

The present invention is directed to a novel annealing control primer system (“ACP system”), for regulating primer annealing specificity during polymerase chain reaction (PCR). The principle of the ACP system is based on the composition of an oligonucleotide primer having 3′- and 5′-end distinct portions separated by a deoxyinosine group, which is a unique feature of this invention. The present invention also provides a process using the ACP system for performing two stage PCR amplifications to selectively amplify a target nucleic acid fragment from a nucleic acid or a mixture. The present invention also provides a method using the ACP system for detecting and cloning differentially expressed mRNAs in two or more nucleic acid samples. Kits containing ACP are included within the scope of the present invention. Furthermore, the present invention can be adapted to almost unlimited application in all fields of PCR-based technology.

This application claims the benefit of PCT/KR01/02133 filed Dec. 8, 2001.

### FIELD OF THE INVENTION

This present invention relates to a novel annealing control primer system, named ACP system, for regulating primer annealing specificity during PCR. This invention allows performing two-stage PCR amplifications to selectively amplify a target nucleic acid sequence from a nucleic acid or a mixture of nucleic acids. This present invention also can be adapted to almost unlimited application in all fields of PCR-based technology.

### BACKGROUND OF THE INVENTION

The method known as polymerase chain reaction (PCR), is based on repeated cycles of denaturation of double-stranded DNA, followed by oligonucleotide primer annealing to the DNA template, and primer extension by a DNA polymerase (Mullis et al. U.S. Pat. Nos. 4,683,195, 4,683,202, and 4,800,159; Saiki et al. 1985). The oligonucleotide primers used in PCR are designed to anneal to opposite strands of the DNA, and are positioned so that the DNA polymerase catalyzed extension product of one primer can serve as the template strand for the other primer. The PCR amplification process results in the exponential increase of discrete DNA fragments whose length is defined by the 5′ ends of the oligonucleotide primers.

One critical parameter for successful amplification in a PCR is the correct design of the oligonucleotide primers. Thus, several parameters such as primer length, annealing temperature, GC content, and PCR product length should be considered in primer design (Dieffenbach et al., 1995). Well-designed primers can help avoid the generation of background and nonspecific products as well as distinguish between cDNA or genomic templates in RNA-PCR. Primer design also greatly affects the yield of the products. When poorly designed primers are used, no or very little product is obtained, whereas correctly designed primers generate an amount of product close to the theoretical values of product accumulation in the exponential phase of the reaction.

Considering the effect of parameters of primer design, such as primer length, annealing temperature, GC content, and PCR product length issued above, there remains a need for a universal primer design that is relatively less sensitive to such parameters.

For solving the design problems associated with the degenerate primers, nucleotides at some ambiguous positions of degenerate primers can be substituted by universal base or a non-discriminatory analogue such as deoxyinosine (Ohtsuka et al, 1985; Sakanari et al., 1989), 1-(2′-deoxy-beta-D-ribofuranosyl)-3-nitropyrrole (Nichols et al., 1994), or 5-Nitroindole (Loakes and Brown, 1994) because such universal bases are capable of non-specifically base pairing with all four conventional bases.

By utilizing the property of universal base, it should be possible that universal bases present in a primer could generate low annealing temperature during PCR because of their weaker hydrogen bonding interactions in base paring. This simple theory leads us to apply universal bases to the design of annealing control primers described in this invention.

In many cases the primer sequence does not need to be a perfect complement to the template sequence. The region of the primer that should be perfectly matched to the template is the 3′-end because this is the end of the primer that is extended by the DNA polymerase and is therefore most important for ensuring the specificity of annealing to the correct target sequence. The 5′-end of the primer is less important in determining specificity of annealing to the target sequence and can be modified to carry additional sequence, such as restriction sites or promoter sequences that are not complementary to the template (McPherson and Moller 2000). This notion is also adapted to the design of annealing control primers described in this invention.

The present invention provides a novel annealing control primer system, named ACP system, for regulating primer annealing during PCR and this ACP system allows enhancing the specificity of primer annealing and the efficiency of amplification.

PCR based techniques have been widely used not only for amplification of a target DNA sequence but also for scientific applications or methods in the fields of biological and medical research (Mcpherson and Moller, 2000).

Techniques designed to identify genes that are differentially regulated by cells under various physiological or experimental conditions (for example, differentiation, carcinogenesis, pharmacologic treatment) have become pivotal to modem biology. One such method for screening differences in gene expression between various cell types or between different stages of cell development with the availability of PCR is known as Differential Display PCR (DD-PCR), described by Liang and Pardee in 1992. This method uses combinations of 10-mer arbitrary primers with anchored cDNA primers and generates fragments that originate mostly from the poly(A) tail and extend about 50-600 nucleotide upstream. By combining 3′ anchored oligo(dT) primers and short 5′ arbitrary primers, subsets of the transcriptome are amplified, the resulting cDNA fragments are separated on denaturing polyacrylamide gel and visualized autoradiographically.

Although this method is simple and rapid and only requires small amounts of total RNA, there are a number of disadvantages to the previous DD-PCR methods. The differential banding patterns are often only poorly reproducible due to the use of short arbitrary primer so that many laboratories have had difficulty obtaining reproducible results with these methods. It has been shown that at least 40% of the differentially displayed bands are not reproducible between experiments even in experienced hands (Bauer el al., 1994). Furthermore, the pattern of differential expression often cannot be reproduced on Northern blots and the percentage of false positives can be as high as 90% (Sompayrac et al., 1995). As a modification used for an alternative, the use of longer random primers of, e.g., 20 bases in length does not satisfactorily solve the problem of reproducibility (Ito et al., 1994). There are other factors leading to the relatively low reproducibility of DD-PCR such as an insufficient amount of starting material and very low concentration of dNTP (2-5 μM) used to prepare the different banding patterns (Matz and Lukyanov, 1998). It is also difficult to detect rare transcripts with these methods (Matz and Lukyanov, 1998). In addition, because the cDNA fragments obtained from DD-PCR are short (typically 100˜500 bp) and correspond to the 3′-end of the gene that represent mainly the 3′ untranslated region, they usually do not contain a large portion of the coding region. Therefore, labor-intensive full-length cDNA screening is needed unless significant sequence homology, informative for gene classification and prediction of function, is obtained (Matz and Lukyanov, 1998).

Differential Display methods use radioactive detection techniques using denaturing polyacrylamide gels. The radioactive detection of the reaction products restricts the use of this technique to laboratories with the appropriate equipment. Relatively long exposure times and problems with the isolation of interesting bands from the polyacrylamide gels are additional drawbacks of Differential Display technique. Although modified non-radioactive Differential Display methods have recently been described, which include silver staining (Gottschlich et al. 1997; Kociok et al., 1998), fluorescent-labeled oligonucleotides (Bauer et al. 1993; Ito et al. 1994; Luehrsen et al., 1997; Smith et al., 1997), the use of biotinylated primers (Korn et al., 1992; Tagle et al., 1993; Rosok et al., 1996), and ethidium bromide-stained agarose gels (Rompf and Kahl, 1997; Jefferies et al., 1998; Gromova et al., 1999), these methods have met with only limited success. If the reaction products could be simply detected on ethidium bromide-stained agarose gel and the results were reproducible and reliable, it would greatly increase the speed and avoid the use of radioactivity.

The present invention provides an improved method, using the ACP system of this invention, to overcome the problems and limitations associated with the previous Differential Display methods described above in detecting differentially expressed mRNAs.

### SUMMARY OF THE INVENTION

The present invention is directed to a novel annealing control primer system, referred to herein as the ACP system, for regulating primer annealing specificity during polymerase chain reaction (PCR). The principle of the ACP system is based on the composition of an oligonucleotide primer having 3′- and 5′-end distinct portions separated by at least one deoxyinosine group. According to the property of deoxyinosine as universal base, the presence of one or more deoxyinosine groups between the 3′- and 5′-end portions of ACP associated with a particular annealing temperature can limit primer annealing to the 3′-end portion only, and block annealing of the 5′-end portion at the particular annealing temperature. It has been discovered by this invention that the presence of at least one deoxyinosine residue group between the 3′- and 5′-end portions of ACP is capable of differentially controlling the annealing of the 5′-end portion sequence to the template through alteration of the annealing temperature, while the 3′-end portion sequence is consistently involved in annealing to the template. In addition, the presence of at least one deoxyinosine residue group immediately 5′ to the 3′-end portion sequence of ACP can also affect the annealing temperature of the 3′-end portion sequence. As a result, an oligonucleotide primer containing a universal base group such as a deoxyinosine residue group between the 3′- and 5′-end portions thereof can be involved in two different occasions of primer annealing because the deoxyinosine residue group acts as a regulator in controlling primer annealing associated with any particular annealing temperature during PCR.

The present invention also provides a process using the ACP system for performing two stage PCR amplifications to selectively amplify a target nucleic acid sequence of a nucleic acid or present in a mixture of nucleic acids.

The present invention also provides a method using the ACP system for detecting and cloning differentially expressed mRNAs in two or more nucleic acid samples.

In addition, the present invention can be used for detecting polymorphisms in genomic fingerprinting.

The present invention also can be used for the isolation of unknown DNA sequences with degenerate primers.

The invention may further be useful in general PCR procedures associated with parameters of primer design, comprising primer length, annealing temperature, GC content, and PCR product length.

The invention may further be also useful for analyzing specific nucleic acid sequences associated with medical diagnostic applications such as infectious diseases, genetic disorders or cellular disorders such as cancer, as well as amplifying a particular nucleic acid sequence.

Kits containing ACP are within the scope of the present invention.

The present invention also can be adapted to almost unlimited application in all fields of PCR-based technology.

### BRIEF DESCRIPTION OF THE DRAWINGS

FIGS. 1A and 1B show a schematic representation of the novel ACP system for performing two stage PCR amplifications according to the subject invention.

FIG. 2 shows a schematic representation as applied to the identification of differentially expressed genes according to the subject invention.

FIG. 3 is a photograph of an agarose gel to show the effect of a deoxyinosine group positioned between the 3′- and 5′-end portions of ACP. The cDNA was amplified using total RNA isolated from fetal tissues at E4.5 (lanes 1 and 4), E11.5 (lanes 2 and 5), and E18.5 (lanes 3 and 6), with a set of the dT₁₀-JYC2 (SEQ ID NO. 29) and ACP10 (lanes 1-3) (SEQ ID NO. 13), and a set of the dT₁₀-ACP1 (SEQ ID NO. 30) and ACP10 (lanes 4-6), respectively.

FIGS. 4A and B and 5A and B are photographs of agarose gels to show examples of the ACP system used for detecting differentially expressed mRNAs during embryonic development using different stages of mouse fetal tissues. The cDNAs were amplified using total RNA isolated from fetal tissues at E4.5 (lane 1 of FIG. 4A, lanes 1-2 and 7-8 of FIG. 4B), E11.5 (lane 2 of FIG. 4A, lanes 3-4 and 9-10 of FIG. 4B), and E18.5 (lane 3 of FIG. 4A, lanes 5-6 and 11-12 of FIG. 4B), with a set of ACP3 (SEQ ID NO. 3) and dT₁₀-ACP1 (FIG. 4A), a set of ACP5 (SEQ ID NO. 5) and dT₁₀-ACP1 (the lanes 1-6 of FIG. 4B), and a set of ACP8 (SEQ ID NO. 8) and dT₁₀-ACP1 (lanes 7-12 of FIG. 4B), respectively. A set of the ACP10 (SEQ ID NO. 13) and dT₁₀-ACP1 (FIG. 5A) and a set of ACP14 (SEQ ID NO. 17) and dT₁₀-ACP1 (FIG. 5B) were used for detecting differentially expressed mRNAs using total RNA isolated from fetal tissues at E4.5 (lanes 1-2 of FIGS. 5A and 5B), E11.5 (lanes 3-4 of FIGS. 5A and 5B), and E18.5 (lanes 5-6 of FIGS. 5A and 5B). The bands indicated by arrows represent the cDNA fragments amplified from differentially expressed mRNAs. The numbers of the arrows indicate the cDNA fragments used as probes in the Northern blot analysis of FIG. 6.

FIG. 6A-F shows Northern blot analysis of six cDNA fragments amplified from differentially expressed mRNAs during embryonic development. The six 32P-labeled fragments indicated by arrows in FIG. 4 were used as probes for Northern blot analysis. The arrows 1, 2, 3, 4, 5, and 6 are DEG1 (FIG. 6A), DEG3 (FIG. 6B), DEG2 (FIG. 6C), DEG8 (FIG. 6D), DEG5 (FIG. 6E), and DEG7 (FIG. 6F), respectively, wherein the results of the DEG sequence analysis are shown in Table 1. DEG2 (SEG ID NO. 31) and DEG5 (SEG ID NO. 32) are turned out as novel genes (Table 2). The control panels (the lower part of each panel) show each gel before blotting, stained with ethidium bromide and photographed under UV light, demonstrating similar levels of 18S and 28S rRNA as a loading control.

FIG. 7 shows the expression patterns of a novel gene, DEG5, in a full stage of mouse fetal. Northern blot analysis was performed using the radiolabeled DEG5 cDNA fragment as a probe. Total RNA (20 μg/lane) was prepared from mouse fetuses at the gestation times as indicated. The control panel at the lower part shows a gel before blotting, stained with ethidium bromide and photographed under UV light, demonstrating similar levels of 18S and 28S rRNA as a loading control.

### DETAILED DESCRIPTION OF THE INVENTION

The present invention is directed to a novel annealing control primer system, named ACP system, for regulating primer annealing specificity during polymerase chain reaction (PCR). The principle of the ACP system is based on the composition of an oligonucleotide primer having 3′- and 5′-end distinct portions separated by at least one deoxyinosine group. According to the property of deoxyinosine as universal base, the presence of a deoxyinosine group between the 3′- and 5′-end portions of ACP associated with a particular annealing temperature can limit primer annealing specifically to the 3′-end portion, not to the 5′-end portion. It has been discovered by this invention that the presence of at least one deoxyinosine residue group between the 3′- and 5′-end portions of ACP is capable of controlling the annealing of the 5′-end portion sequence to the template depending on alteration of annealing temperature, while the 3′-end portion sequence is consistently involved in annealing to the template. In addition, the presence of at least one deoxyinosine residue group immediately 5′ to the 3′-end portion sequence of ACP can also alter the annealing temperature of the 3′-end portion sequence. As a result, an oligonucleotide primer containing a universal base group such as a deoxyinosine residue group or groups between the 3′-end and 5′-end portions thereof can be involved in two different occasions of primer annealing because the deoxyinosine residue group acts as a regulator in controlling primer annealing in associated with annealing temperature during PCR.

A deoxyinosine group positioned between the 3′- and 5′-end portions of ACP described herein is designed to define each portion.

The term “template” refers to nucleic acid molecule. The term “portion” refers to a nucleotide sequence flanked by at least one deoxyinosine residue group, universal base or non-discriminatory base analog. The term “3′-end portion” or “5′-end portion” refers to a nucleotide sequence at the 3′ end or 5′ end of a primer, respectively, which is flanked by at least one deoxyinosine residue group, universal base or non-discriminatory base analog.

The term “primer” as used herein refers to an oligonucleotide, whether occurring naturally or produced synthetically, which is capable of acting as a point of initiation of synthesis when placed under conditions in which synthesis of primer extension product which is complementary to a nucleic acid strand (template) is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and source of primer.

The term “annealing” or “priming” as used herein refers to the apposition of an oligodeoxynucleotide or nucleic acid to a template nucleic acid, whereby said apposition enables polymerase to polymerize nucleotides into a nucleic acid molecule which is complementary to the template nucleic acid or a portion thereof.

The term “substantially complementary” is used herein to mean that the ACP and a target sequence share sufficient nucleotide similarities to enable annealing of the ACP to the target sequence under the designated annealing conditions, such that the annealed primer can be extended by polymerase to form a complementary copy of the template.

The term “normal cell” is used to mean any cell that is not in a diseased or pathologic state.

The ACP system in this invention is significantly effective and widely accessible to PCR based applications. Also, various problems related to primer annealing specificity remaining for the previous PCR techniques can be solved by the ACP system. The main benefits to be obtained from the use of the ACP system are as follows:

(a) primer annealing can be controlled because a deoxyinosine residue group between the 3′- and 5′-end portions of ACP can limit primer annealing to the 3′-end portion only through alteration of annealing temperature during PCR amplification, which results in improving the specificity of primer annealing during PCR. This is the fundamental difference between the ACP-PCR and the previous general PCR: the ACP-PCR allows having the two stages of amplifications, whereas the general PCR has only one stage PCR amplification.

(b) two stage PCR amplifications can be performed at low and high stringent conditions, respectively, which enables to selectively amplify a target nucleic acid fragment from a nucleic acid or a mixture.

(c) mispriming which is a major cause of false product amplification during PCR can be significantly minimized.

(d) the efficiency of PCR amplification is increased, which makes it easier to detect rare mRNAs.

(e) the reproducibility of PCR products is increased, which saves a great amount of time and cost.

(f) agarose gel electrophoresis followed by ethidium bromide staining can be used for detecting differentially displayed RT-PCR products.

The present invention provides an improved process using the ACP system for performing two stage PCR amplifications to selectively amplify a target nucleic acid fragment from a nucleic acid or a mixture, wherein the process comprises the following steps:

(1) amplifying a target nucleic acid fragment present in a nucleic acid or a mixture by a first stage PCR using ACPs under conditions such that the 3′-end portion of each ACP anneals to a site of the template at a first annealing temperature and wherein the 5′-end portion serves as a universal priming site for subsequent amplification of a first PCR product generated from the 3′-end portion of each ACP, wherein amplification is carried out by at least one cycle of the denaturing, annealing and primer extension steps of PCR to obtain amplification products; and

(2) re-amplifying the first PCR product generated from step (1) at high stringent conditions by a second stage PCR using the universal sequences of the 5′ end portion of the ACP as primers. The first PCR products generated from step (1) contain ACP sequences at their 5′ ends and thus, the 5′-end portion sequences of ACPs are used as universal primer sequences in step (2).

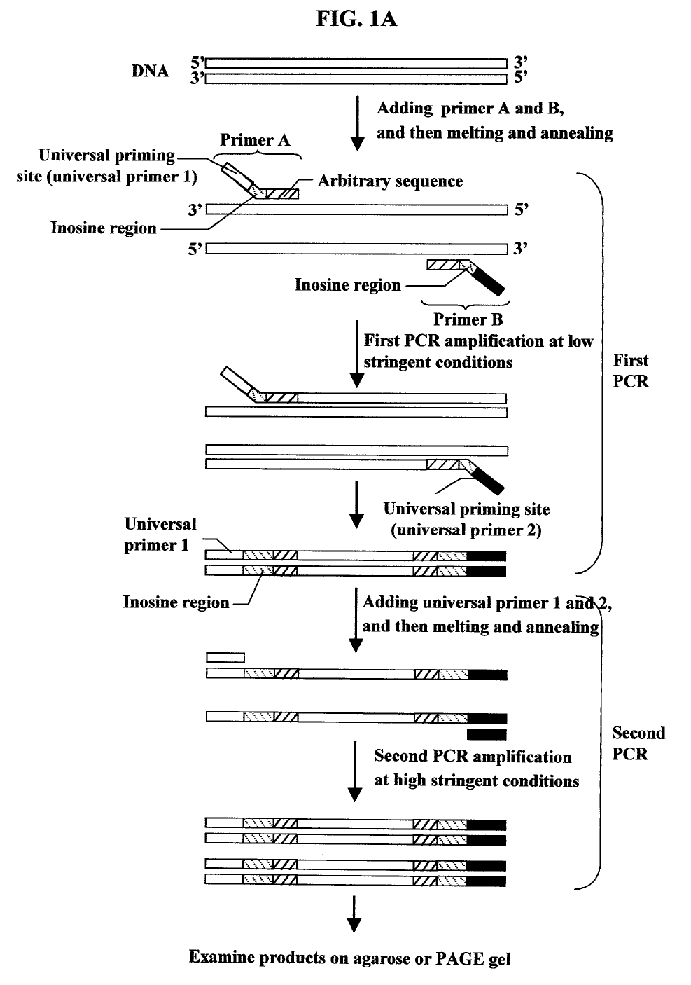

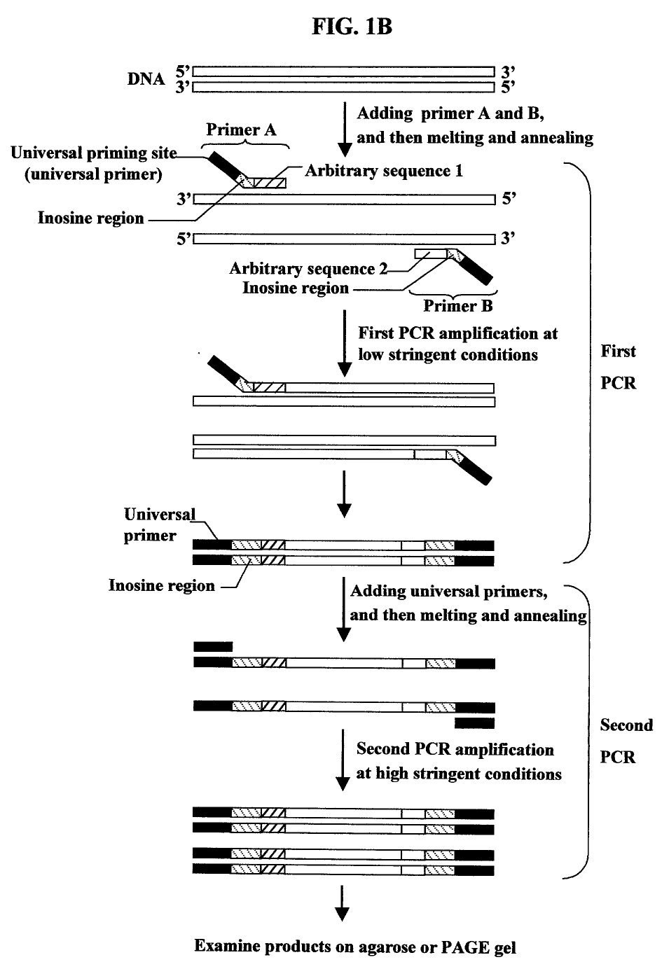

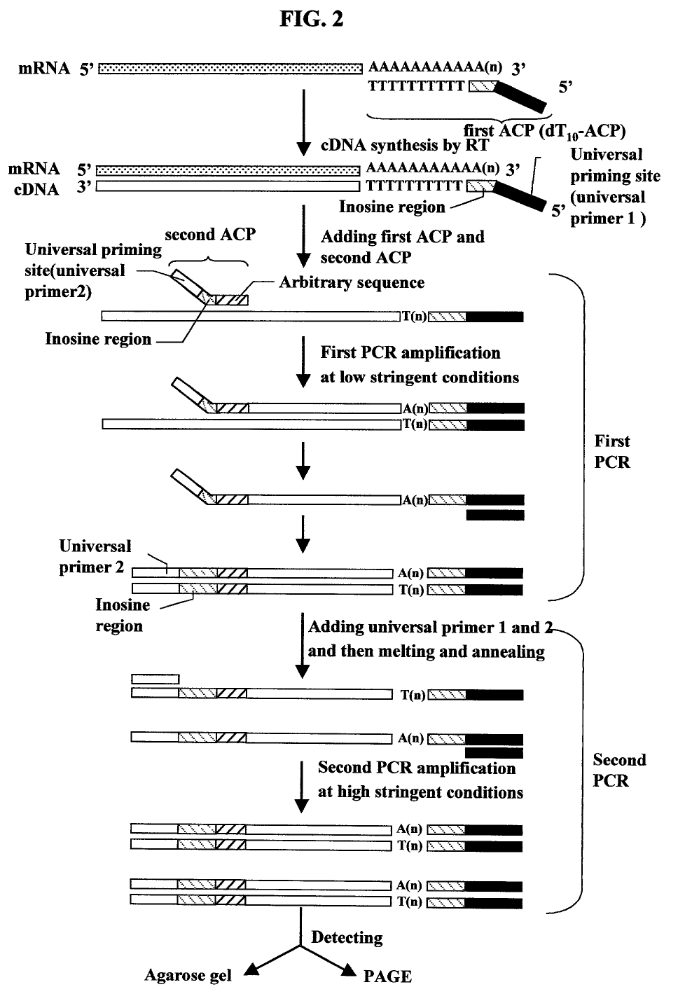

A schematic representation of the novel ACP system for performing two stage PCR amplifications as described above is illustrated in FIG. 1.

In a preferred embodiment, the annealing temperature ranges from 40° C. to 55° C. for the first PCR amplification in step (1). The annealing temperature ranges from 50° C. to 70° C. for the second PCR amplification in step (2). The length of the 3′ end portion sequence of ACP will determine the annealing temperature for the first PCR amplification in step (1). For example, in case that ACP comprises 10 arbitrary nucleotides at the 3′-end portion, preferably, annealing temperature will be about 50° C. for the first PCR amplification in step (1).

In another embodiment, the first PCR amplification under low stringent conditions used in step (1) is carried out for at least 1 cycle of PCR, and through the subsequent cycles, the amplification is processed more effectively under high stringent conditions used in step (2). The first amplification can be carried out up to 30 cycles of PCR. In another embodiment, the second PCR amplification under high stringent conditions used step (2) is carried out for at least 10 cycles and up to 40 cycles of PCR to improve the specificity of primer annealing during PCR. High and low stringency conditions are standard in the art.

“Cycle” refers to the process which results in the production of a copy of target nucleic acid. A cycle includes a denaturing step, an annealing step, and an extending step.

The invention particularly concerns the embodiments of the ACP system as used in the above method, wherein ACP is represented by the following formula (1):

5′-dN₁-dN₂- . . . dNₓ-dI₁-dI₂- . . . dI_{y}-dN₁-dN₂- . . . dN_{z}-3′

wherein dN is one of the four deoxyribonucleotides, A, C, G, or T; dI is a deoxyinosine and the deoxyinosine group is responsible for the main function of ACP in associated with alteration of annealing temperature during PCR; x, y, and z represent an integer, respectively and z should be less than x; dNₓ represents the 5′-end portion and contains a pre-selected arbitrary nucleotide sequence; dI_{y} represents a deoxyinosine region and contains at least 3 deoxyinosines; dN_{z} represents the 3′-end portion.

In a preferred embodiment, each ACP contains at least 3 deoxyinosine residues between the 3′- and 5′-end portion sequences of ACP. Preferably, the deoxyinosine residues between the 3′- and 5′-end portion sequences can be up to 10 deoxyinosine residues in length. Most preferably, the deoxyinosine residues between the 3′- and 5′-end portion sequences are 5 deoxyinosine residues in length. The use of at least one and preferably three deoxyinosine residues between the 3′- and 5′-end portion sequences is a key feature of the present invention because it provides each portion (3′- and 5′-) with a distinct annealing specificity in association with an annealing temperature during PCR.

In one aspect of the invention, a minimum number of linked deoxyinosine residues between the 3′- and 5′-end portions of ACP is preferred in order to interrupt the annealing of the 5′-end portion to the template during PCR at a first annealing temperature. The length of linked deoxyinosine in the sequence (7-10 bases) does not make a significant difference on the effect of deoxyinosine residues in ACP.

In another preferred embodiment, the deoxyinosine residue group responsible for the main function of ACP in association with the alteration of annealing temperature during PCR described herein can be replaced with a non-discriminatory base analogue or universal base group, such as a group of 1-(2′-deoxy-beta-D-ribofuranosyl)-3-nitropyrrole or 5-Nitroindole (Nichols et al., 1994; Loakes and Brown, 1994).

The “preferred length” of an oligonucleotide primer, as used herein, is determined from desired specificity of annealing and the number of oligonucleotides having the desired specificity that are required to hybridize to the template. For example, an oligonucleotide primer of 20 nucleotides is more specific than an oligonucleotide primer of 10 nucleotides because the addition of each nucleotide increases the annealing temperature of the primer to the template.

The lengths of the 3′- and 5′-end portion sequences of the ACP described herein will depend on the objective of each experiment.

In a preferred embodiment, the 3′-end portion of the ACP is at least 6 nucleotides in length, which is a minimal requirement of length for primer annealing. Preferably, the 3′-end portion sequence can be up to 20 nucleotides in length.

In another preferred embodiment, the 5′-end portion of ACP contains at least 15 nucleotides in length, which is a minimal requirement of length for annealing under high stringent conditions. Preferably, the 5′-end portion sequence can be up to 40 nucleotides in length. More preferably, the 5′-end portion sequence is from 20 to 30 nucleotides in length. The entire ACP is, preferably, at least about 35 nucleotides in length, and can be up to about 50 nucleotides in length. The 5′-end portion of ACP has a pre-selected arbitrary nucleotide sequence and this nucleotide sequence is used as a universal primer sequence for subsequent amplification. Using a longer arbitrary sequence (about 22 to 40 bases) at the 5′-end portion of ACP reduces the efficiency of ACP, but shorter sequences (about 15 to 17 bases) reduce the efficiency of annealing at high stringent conditions of ACP. It is also a key feature of the present invention to use a pre-selected arbitrary nucleotide sequence at the 5′-end portion of ACP as a universal primer sequence for subsequent amplification.

A variety of DNA polymerases can be used during PCR with the subject invention. Preferably, the polymerase is a thermostable DNA polymerase such as may be obtained from a variety of bacterial species, including Thermus aquaticus (Taq), Thermus thermophilus (Tth), Thermus filiformis, Thermis flavus, Thermococcus literalis, and Pyrococcus furiosus (Pfu). Many of these polymerase may be isolated from bacterium itself or obtained commercially. Polymerase to be used with the subject invention can also be obtained from cells which express high levels of cloned genes encoding polymerase.

The subject invention can be particularly used for detecting and cloning DNAs complementary to differentially expressed mRNAs in two or more nucleic acid samples using the ACP system. A schematic representation of the subject invention as applied to the identification of differentially expressed genes is illustrated in FIG. 2. The method comprises the following steps of:

(a) providing a first sample of nucleic acids representing a first population of mRNA transcripts and a second sample of nucleic acids representing a second population of mRNA transcripts;

(b) contacting each of the first nucleic acid sample and the second nucleic acid sample with a first ACP, wherein the first ACP has a hybridizing sequence sufficiently complementary to a region of the first and second population of mRNA transcripts to hybridize therewith;

(c) reverse transcribing the mRNA to which the first ACP hybridizes to produce a first population of DNA strands that are complementary to the mRNAs in the first nucleic acid sample to which the first ACP hybridizes, and a second population of DNA strands that are complementary to the mRNA in the second nucleic acid sample to which the first ACP hybridizes;

(d) purifying and quantifying the complementary DNA strands produced as a result of the reverse transcription step (c);

(e) annealing extending a second annealing control primer using DNA polymerase to produce a second DNA strand complementary to the first and second populations of DNA strands;

(f) contacting each of the first and second populations of DNA strands with a second ACP, wherein the second ACP has a hybridizing sequence sufficiently complementary to a region in the first and second populations of DNA strands;

(g) first-amplifying each mixture obtained from step (f) through at least one cycle of the denaturing, annealing and primer extension steps of PCR to obtain amplification products;

(h) second-amplifying the products generated from step (g) using two universal primers each comprising a sequence corresponding to each 5′-end portion of the first and second ACPs; and

(i) comparing the presence or level of individual amplification products in the first and second populations of amplification products.

The method of this invention for detecting differences in gene expression uses only a single cDNA synthesis primer, the first ACP, to react with mRNA, in contrast to multiple cDNA synthesis anchor primers required by a Differential Display PCR. In the original differential display method outlined by Liang and Pardee in 1992, twelve anchor primers were introduced. The anchor primers for example, having a sequence of T_{12 MN}, where M is A, C, or G and N is A, C, G or T, produced twelve separate cDNA populations. Recently, modified anchor primers have been proposed by altering the number of nucleotides such as one or three instead of two at the 3′-end which can hybridize to a sequence that is immediately 5′ to the poly A tail of mRNAs or by extending additional nucleotides at the 5′-end while retaining the oligo(dT)_{9-12 MN} tail resulting in at least 21 nucleotides in length (Villeponteau et al., 1996, Combates et al., 2000).

This invention particularly concerns the embodiments of the ACP system as used in the above method, wherein the first ACP used in step (b) is represented by the following general formula (2):

5′-dN₁-dN₂- . . . dNₓ-dI₁-dI₂- . . . dI_{y}-dT₁-dT₂- . . . dT-3′

wherein dN is one of the four deoxyribonucleotides, A, C, G, or T; dI is a deoxyinosine and the deoxyinosine group is responsible for the main function of the ACP associated with alteration of annealing temperature during PCR; dT is a T deoxyribonucleotide; x, y, and z represent an integer, respectively and z should be less than x; dNₓ represents the 5′-end portion and contains a pre-selected arbitrary nucleotide sequence; dI_{y} represents a deoxyinosine region and contains at least 3 deoxyinosines; dT_{z} represents the 3′-end portion.

The above formula (2) basically follows the rule of formula (1) except at the 3′-end portion of ACP. The 3′-end portion of formula (2) contains a sequence capable of annealing to the poly A tail of mRNA and serves as a cDNA synthesis primer for reverse transcription of mRNA.

In a preferred embodiment, the 3′-end portion of the first ACP used in step (b) contains at least 6 T nucleotides in length, which is a minimal requirement of length for primer annealing. Preferably, the 3′-end portion sequence can be up to 20 T nucleotides in length. Most preferably, the 3′-end portion sequence is about 10 T nucleotides in length. This primer is named dT₁₀ annealing control primer (dT₁₀-ACP). In some embodiments, the 3′-end portion of the first ACP used in step (b) may contain at least 1 additional nucleotide at the 3′ end that can hybridize to an mRNA sequence which is immediately upstream of the polyA tail. The additional nucleotides at the 3′ end of the first ACP may be up to 3 in length. The additional polyA-non-complementary nucleotides are of the sequence M, MN, or MNN, where M can be G (guanine), A (adenine), or C (cytosine) and N can be G, A, C, or T (thymidine). Most preferably, the 3′-end portion sequence of the first ACP used in step (b) contains dT₁₀ only.

In a preferred embodiment, the 5′-end portion of the first ACP used in step (b) contains at least 15 nucleotides in length, which is a minimal requirement of length for high stringent conditions. Preferably, the 5′-end portion sequence can be up to 40 nucleotides in length. Most preferably, the 5′-end portion sequence is about 22 nucleotides in length. Using a longer arbitrary sequence (about 25 to 40 bases) at the 5′-end portion of the first ACP reduces the efficiency of the first ACP, but shorter sequences (about 15 to 17 bases) reduce the efficiency of high stringent conditions for increasing the specificity of primer annealing during PCR. It is another key feature of the present invention to use a pre-selected arbitrary nucleotide sequence at the 5′-end portion of the first ACP as a universal primer sequence for subsequent amplification.

In a preferred embodiment, the first ACP also contains at least 3 deoxyinosines between the 3′- and 5′-end portion sequences. Preferably, the deoxyinosine residues between the 3′- and 5′-end portion sequences can be up to 10 deoxyinosines in length. Most preferably, the deoxyinosine residues between the 3′- and 5′-end portion sequences are 5 deoxyinosines in length. The first entire ACP is preferably at least 35 nucleotides in length, and can be up to 50 nucleotides in length.

The first ACP described herein is hybridized to the poly A tail of the mRNA, which is present on all mRNAs, except for a small minority of mRNA. The use of the first ACP used in this invention results in only one reaction and produces only one cDNA population, in contrast to at least 3 to 64 separate cDNA populations generated by anchor primers of Differential Display technique. This greatly increases the efficiency of the method by generating a substantially standard pool of single-stranded cDNA from each experimental mRNA population.

In the step (d), the standard pools of cDNAs synthesized by the first ACP should be purified and then quantitated by spectrophotometry, in accordance with techniques well-known to those of ordinary skill in the art. This step is necessary to precisely control their input into the PCR amplification step and then compare the final PCR products between two or more samples. Preferably, the amount of cDNA produced at this point in the method is measured. Preferably, this determination is made using ultraviolet spectroscopy, although any standard procedure known for quantifying cDNA known to those of ordinary skill in the art is acceptable for use for this purpose. When using the UV spectroscopy procedure, an absorbance of about 260 nm of UV light preferably is used. By the measurement of cDNA quantity at this step, therefore, the cDNA quantity can be standardized between or among samples in the following PCR reaction.

After synthesis of cDNAs using the first ACP, the resultant cDNAs are then amplified by the two stage PCR amplifications using the ACP system described herein. The first PCR amplification of cDNA is carried out under low stringent conditions using the first and second ACPs used in steps (b) and (e) as 5′ and 3′ primers, respectively.

In a preferred embodiment, the second ACP used in step (e) contains a short arbitrary sequence at the 3′-end portion and this primer is named an arbitrary annealing control primer (ARACP). Preferably, the ARACP can have from 8 to 13 nucleotides in length at the 3′ end. Most preferably, ARACP contains about 10 nucleotides in length at the 3′ end. The formula for ARACP is identical to the formula (1).

In a preferred embodiment, the 5′-end portion of ARACP used in step (e) contains at least 15 nucleotides in length. Preferably, the 5′-end portion sequence can be up to 40 nucleotides in length. Most preferably, the 5′-end portion sequence is about 22 nucleotides in length. The entire ARACP is preferably at least 35 nucleotides in length, and can be up to 50 nucleotides in length. Like the 5′-end portion sequence of the first ACP, the 5′-end portion contains a pre-selected arbitrary sequence and will be used as a universal priming site. However, the 5′-end portion sequence of ARACP used in step (e) should be different from that of the first ACP used in step (b).

ARACP described herein is different from a so-called long arbitrary primer, as used in the known modified Differential Display technique. For example, the conventional long arbitrary primers as described by Villeponteau et al. (1996) and Diachenko et al. (1996), having at least 21 or 25 nucleotides in length, comprise of only arbitrary nucleotides in the entire sequences. Thus, these conventional long arbitrary primers under the low annealing temperature (about 40° C.) required in the early PCR cycle to achieve arbitrary priming will hybridize in a non-predictable way, making a rational design of a representative set of primers impossible. Furthermore, many of the bands represent the same mRNA due to the “Stickiness” of long primers when used under such a low stringency.

One of significant embodiments of the present invention is the use of ARACP for detecting differences in gene expression. Since ARACP is designed to limit the annealing of ARACP to the 3′-end portion sequence, not to the 5′-end portion sequence in association with annealing temperature, the resultant annealing will come out in a predictable way, making a rational design of a representative set of primers possible. In addition, ARACP system allows avoiding false positive problems caused by the “Stickiness” of the conventional long primers under low stringent conditions as used in the previous Differential Display technique.

In a preferred embodiment, the annealing temperature used for the first PCR amplification under low stringency conditions used in step (g) is about between 45° C. and 55° C. Most preferably, the annealing temperature used for the first PCR amplification under low stringency conditions is about 50° C. However, unlike Differential Display, which uses annealing temperatures between 35° C. and 45° C., the annealing temperature of low stringency conditions used herein is relatively higher than those used in the known classical or enhanced Differential Display techniques with arbitrary primers.

Another significant embodiment of the present invention is the use of the first ACP system in the first PCR amplification for detecting differences in gene expression. The annealing of the first ACP is interrupted by the presence of the deoxyinosine residue group between the 3′- and 5′-end portions under relatively high stringent conditions based on the following assumptions:

(i) the presence of a deoxyinosine group in the ACP generates a low annealing temperature at the site of the deoxyinosine region(s), which is an inherent property of deoxyinosine.

(ii) annealing of the 3′-end portion of the ACP may be independent from the 5′-end portion, since the deoxyinosine group separates the 3′-end and 5′-end portions in their annealing due to its weaker hydrogen bonding interactions in base pairing.

(iii) 3′-end portion of ACP acts only as annealing site to the template during PCR.

(iv) Tm of dT₁₀ having 10 T nucleotides is too low for the 10 T nucleotides to bind the template.

(v) consequently, the dT₁₀ will not produce any PCR products under high annealing temperature.

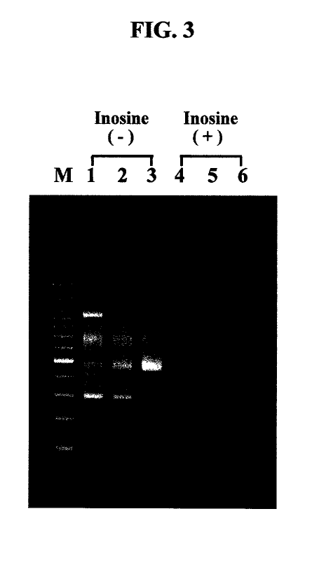

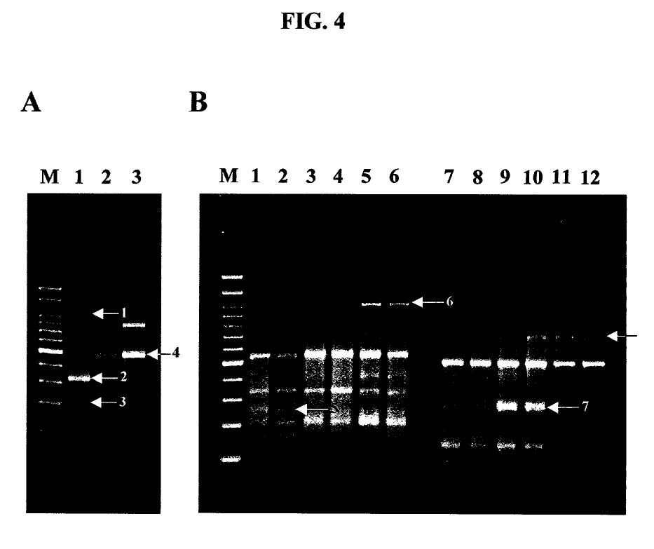

Consistent with the above assumption, FIG. 3 shows that the first ACP (such as dT₁₀-ACP) produces almost no products under such annealing temperature of 54° C., whereas the conventional long oligo dT such as dT₁₀-JYC2, which does not have the deoxyinosine residue group, but contains the same nucleotide at the 5′ end portion, produces a lot of products. These results indicate that the annealing of the 3′-end portion (10 T nucleotides) of the dT₁₀-ACP is independent from the 5′-end portion due to the presence of the deoxyinosine residue group between the 3′- and 5′-end portions under such temperature of 54° C.

On the other hand, when an appropriate annealing temperature is given, for example about 50° C., the first ACP will be annealed selectively to the template sequence, which is perfectly complement to any sequence of the first ACP. For this reason, about 50° C. is determined as a proper annealing temperature for screening gene expression in this invention.

In a preferred embodiment, the first PCR amplification under low stringent conditions used in step (g) is carried out by at least 1 cycle of PCR to achieve arbitrary priming, and through the subsequent cycles, the amplification is processed more effectively under high stringent conditions used in step (h). The first amplification can be carried out by up to 30 cycles of PCR. The cycle of the first PCR amplification can be varied in accordance with the types of samples. For example, 20 cycles of the first PCR amplification were used for mouse fetal samples and 1 cycle was used for soybean shoot samples. An example of the first PCR amplification consisting of 20 cycles at low annealing conditions used in step (g) is conducted under the following conditions: in a final volume of 50 μl containing 50 ng of the first-strand cDNA, 5 μl of 10×PCR reaction buffer (Promega), 3 μl of 25 mM MgCl₂ 2, 5 μl of dNTP (0.2 mM each dATP, dCTP, dGTP, dTTP), 5 μl of 5′ primer (1 μM), 5 μl of 3′ primer (1 μM), and 0.5 μl of Taq polymerase (5 units/μl). An example of the first PCR amplification under low annealing conditions described in step (g) is as follows: 5 min at 94° C., followed by 20 cycles at 94° C. for 1 min, 50° C. for 1 min, and 72° C. for 1 min; followed by a 5 min final extension at 72° C.

In a preferred embodiment, the second PCR amplification of the resultant cDNAs produced by the step (h) is carried out under high stringency conditions using two universal primers each comprising a sequence corresponding to each 5′-end portion of the first and second ACPs. In contrast, the previous Differential Display methods use the same primers for high stringency conditions as well as for low stringency conditions, and thus have the following limitations and drawbacks, namely the high false positive rate and possible under-representation of minor mRNA fractions in the analysis, which are the main problems in the previous Differential Display techniques.

In a preferred embodiment, the annealing temperature of the second PCR amplification for high stringency conditions used in step (h) is preferably about between 55° C. and about 70° C. Most preferably, the annealing temperature used for the high stringent conditions is about 65° C.

In a preferred embodiment, the second PCR amplification under high stringency conditions used step (h) is carried out by at least 10 cycles and up to 40 cycles of PCR to improve the specificity of primer annealing during PCR. Most preferably, the first amplification is carried out by 30 cycles of PCR. An example of the second PCR amplification by 30 cycles under high stringency annealing conditions used in step (h) is conducted at the following conditions: in a final volume of 50 μl containing 5 μl of the first amplified cDNA products (50 μl), 5 μl of 10×PCR reaction buffer (Promega), 3 μl of 25 mM MgCl₂, 5 μl of 2 mM dNTP, 1 μl of 5′ primer (10 EM), 1 μl of 3′ primer (10 μM), and 0.5 μl of Taq polymerase (5 units/μl). The PCR reactions were as follows: 5 min at 94° C. followed by 30 cycles of 94° C. for 1 min, 65° C. for 1 min, and 72° C. for 1 min; followed by a 5 minutes final extension at 72° C.

Another significant embodiment of the present invention is the use of high annealing temperature in a method for detecting differences in gene expression. High annealing temperature used in step (h) increases the specificity of primer annealing during PCR, which results in eliminating false positive products completely and increasing reproducibility. Freedom from false positives which is one major bottleneck remaining for the previous Differential Display technique is especially important in the screening step for the verification of the cDNA fragments identified by Differential Display.

In a preferred embodiment, the resultant PCR cDNA fragments produced by step (h) are separated by electrophoresis to identify differentially expressed mRNAs. Preferably, the resultant PCR cDNA fragments are detected on an ethidium bromide-stained agarose gel. In some embodiment, the resulting PCR cDNA fragments are detected on a denaturing polyacrylamide gel.

Another significant feature of this invention is the use of ethidium bromide-stained agarose gel in the identification of differentially expressed mRNAs. In general, the previous Differential Display methods use radioactive detection techniques using denaturing polyacrylamide gels. The significant amount of the amplified cDNA fragments can be obtained through two stage PCR amplifications described herein, which allows to use an ethidium bromide-stained agarose gel to detect the amplified cDNAs. Thus, the use of ethidium bromide-stained agarose gel results in increasing the speed and avoiding the use of radioactivity.

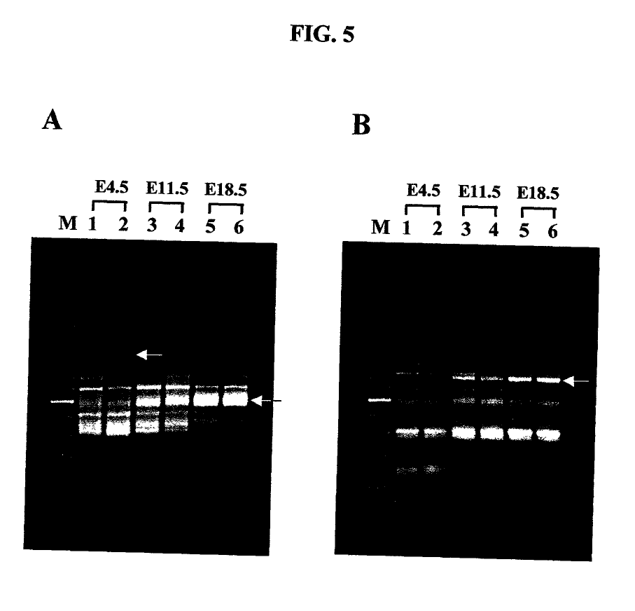

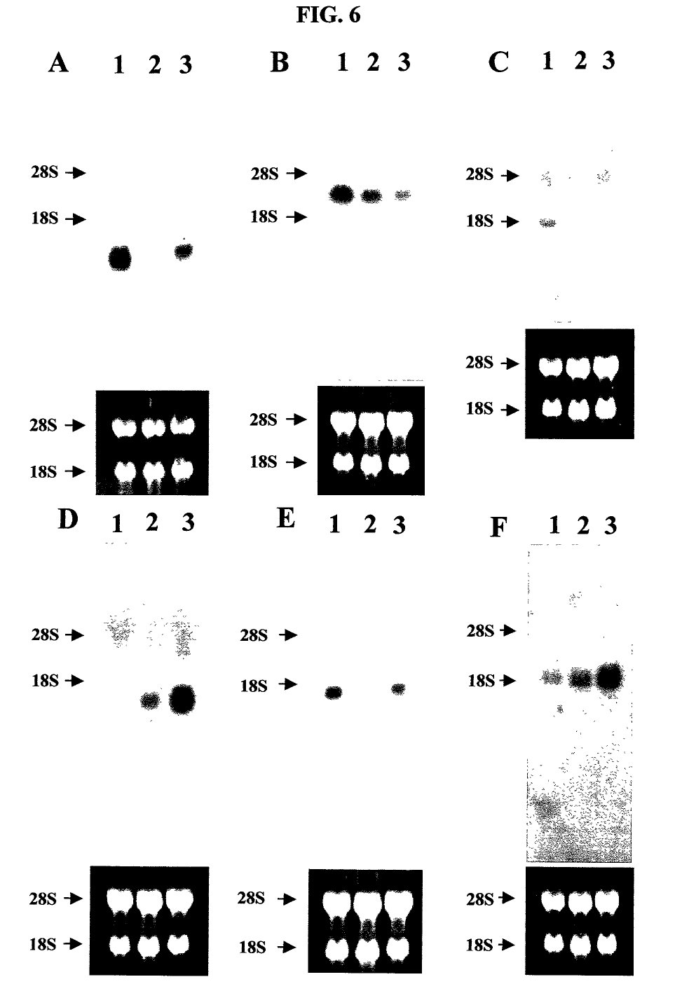

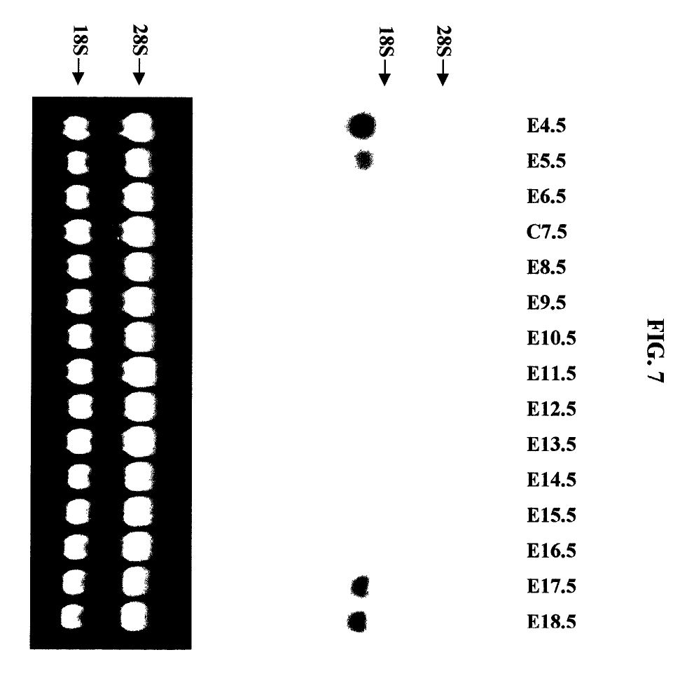

FIGS. 4-5 shows examples of the ACP system used for the analysis of gene expression during embryo development using different stages of mouse fetal tissues. Many bands differentially expressed during development are detected on 2% ethidium-stained agarose gels, cloned into pGEM-T easy vector (Promega), and characterized by sequencing and Northern blot analysis. The sequence analysis reveals that all of the clones are known genes except two novel genes (Table 1). The nucleotide sequences of two novel cDNA fragments, named DEG 2 and DEG 5, are shown in Table 2. Many authentic differentially expressed genes during embryonic development have been discovered by this invention. Surprisingly, consistent with the results of agarose gel analysis, Northern blot analysis showed that the expression patterns of the eleven clones are identical with the original bands on the agarose gels, indicating that all of the clones are positive products (FIGS. 6-7). Thus, the ACP system produces only positive products without any false positives, which means that the ACP system eliminates totally the problem of false positives.

As a result, the method described by this invention for detecting and cloning differentially expressed genes differs from the previous Differential Display techniques in several ways. Fundamentally, the use of the ACP system in this method makes it possible to allow two stages of PCR amplifications and to use the sufficient amount of starting materials as well as the high concentration of dNTP, resulting in the following benefits: a) increasing the specificity of primer annealing, b) eliminating the problem of false positives and avoiding the subsequent labor-intensive work to verify true positives, c) improving reliability and reproducibility, d) detecting rare mRNAs, e) generating large PCR products ranging in size from 150 bp to 1.2 kb, f) allowing the use of ethidium bromide to detect products, g) increasing the speed, h) particularly, not requiring prior experience to conduct this method, i) making a rational design of a representative set of primers possible.

In another embodiment, the ACP method in the subject invention also can be used for detecting polymorphisms in genomic fingerprinting generated by the present ACP method. In the previous arbitrarily primed PCR fingerprints, called AR-PCR, short or long arbitrary primers are used under non-stringent conditions for early 2-5 cycles of PCR amplification because a low annealing temperature is required to achieve arbitrary priming. Although effective amplification proceeds in the following cycles under high stringency condition, false positives still comprise a significant portion of isolated fragments because the same arbitrary primers are used in the following high stringency conditions. For example, the ACP contains an arbitrary sequence at the 3′-end portion with at least 6 nucleotides in length. Preferably, the 3′-end portion contains about 10 nucleotides in length. The formula for ACP used in this method is identical to the formula (1). A single ACP or a pair of ACPs can be used for detecting polymorphisms in genomic fingerprinting. Preferably, a pair of ACPs is used for genomic fingerprinting because a pair of ACPs produces more products than a single arbitrary ACP does.

In another embodiment, the invention using the present ACP system may be useful for the isolation of unknown DNA sequences with degenerate primers. There are two principle approaches to the design of degenerate primers: (a) using peptide sequence data obtained from a purified protein; and (b) using consensus protein sequence data from alignments of gene families. If orthologs of the gene of interest have been cloned from other organisms, or if the gene is a member of a gene family, it will be possible to generate protein sequence alignments. These may reveal appropriate regions for the design of degenerate primers, for example, from consensus sequence of highly conserved regions. Amplifications using degenerate primers can sometimes be problematic and may require optimization. The first parameter is annealing temperature. It is important to keep the annealing temperature as high as possible to avoid extensive nonspecific amplification and a good rule of thumb is to use 55° C. as a starting temperature. In general, it is difficult to keep this rule because degenerate primers should be designed based on amino acid sequences as a precondition. However, the ACP system does not have to satisfy this requirement because the ACP system allows a high annealing temperature such as 65° C. at the second stage of PCR amplification, regardless of primer design.

The subject invention can be also useful in general PCR procedures associated with parameters of primer design, comprising primer length, annealing temperature, GC content, and PCR product length. Considering the effect of these parameters issued above, the ACP described herein is relatively less sensitive to such parameters because the ACP system tolerates these “primer search parameters”.

The subject invention can be also used for analyzing specific nucleic acid sequences associated with medical diagnostic applications, such as infectious diseases, genetic disorders or cellular disorders such as cancer, as well as amplifying a particular nucleic acid sequence.

In a further aspect, the invention comprises a kit for performing the above methods. Such a kit may be prepared from readily available materials and reagents.

The following examples demonstrate the mechanism and utility of this invention. They do not serve to limit the scope of the invention, but merely to illustrate the ways in which the method and compositions of this invention may be performed.

### EXAMPLES

For exemplary purposes, the ACP system of this invention was used to identify and characterize differentially expressed genes during mouse embryonic development. Total RNA was isolated from the entire fetuses at day of 4.5, 11.5, and 18.5 during gestation period.

### Example 1

### Evaluation of Deoxyinosine Effect in ACP System

The effect of deoxyinosine residues positioned between the 3′- and 5′-end portions of ACP was evaluated by RT-PCR using mouse fetal tissues. Total RNA was isolated from the entire fetuses of mouse strain ICR at day of 4.5, 11.5, and 18.5 during gestation period using either Tri-reagent (Sigma), or the LiCl/Urea method (Hogan et al., 1994) as previously described (Chun et al., 1999; Hwang et al., 2000).

A. First-Strand cDNA Synthesis.

dT₁₀-JYC2 5′-GCTTGACTACGATACTGTGCGATTTTTTTTTT-3′ (SEQ ID NO. 29) or dT₁₀-ACP1 5′-GCTTGACTACGATACTGTGCGAIIIIITTTTTTTTTT-3′ (SEQ ID NO. 30) was used as a cDNA synthesis primer.

Three microgram of total RNA and two microliter of 10 μM dT₁₀-JYC2 or dT₁₀-ACP1 were combined in a 20 μl final volume. The solution was heated to 65° C. for 10 minutes, quenched on ice, and microfuged to collect solvent at the bottom. The following components were added sequentially to the annealed primer/template on ice: 0.5 μl (40 U/μl) RNasin ribonuclease inhibitor (Promega), 4 μl 5×reaction buffer (250 mM Tris-HCl, pH 8.3, 375 mM KCl, 15 mM MgCl₂, 50 mM DTT), 5 μl of 2 mM each deoxynucleotide mix (DATP, dCTP, dGTP, dTTP), and 1 μl Moloney-murine leukemia virus (M-MLV) reverse transcriptase (200 U/μl). The reaction mixture was incubated at 37° C. for 90 min, microfuged, and placed on ice. The reaction was stopped by incubation at 94° C. for 2 min. The resultant cDNAs were purified by a spin column (PCR purification Kit, QIAGEN) to remove primers, dNTP, and the above reagents. It is necessary to perform the purification step prior to the determination of the cDNAs concentration using the UV spectroscopy at an absorbance of 260 nm. The cDNAs can be stored at −20° C.

B. cDNA Amplification Using ACPs.

The dT₁₀-ACP1 was used to examine the effect of a deoxyinosine group positioned between the 3′- and 5′-end portions during PCR. A dT₁₀-JYC2 primer lacking a deoxyinosine group was used as a control.

This experiment was conducted based on the following assumptions:

(i) a deoxyinosine group in ACP would generate low annealing temperature at the deoxyinosine region caused by the property of deoxyinosine.

(ii) annealing of the 3′-end portion of ACP could be independent from the 5′-end portion since the deoxyinosine group separates the 3′-end portion from the 5′-end portion in its annealing due to its weaker hydrogen bonding interactions in base pairing.

(iii) the 3′-end portion of ACP acts only as annealing site to the template during PCR.

(iv) Tm of dT₁₀ having 10 T nucleotides is too low for the 10 T nucleotides to bind the template.

(v) consequently, the dT₁₀ would not produce any PCR products under high annealing temperature.

The ACP10 5′-GTCTACCAGGCATTCGCTTCATIIIIIGCCATCGACC-3′ (SEQ ID NO. 13) was used as 5′ primer for this experiment.

The PCR amplification was conducted in a 50 μl volume containing 50 ng of the first-strand cDNA, 5 μl 10×PCR buffer, 1 μl 10 μM 5′primer (ACP10), 1 μl 10 μM 3′primer (dT₁₀-JYC2 or dT₁₀-ACP1), 3 μl 25 mM MgCl₂, 5 μl 2 mM dNTP, 0.5 μl Taq polymerase (5 units/μl). The PCR reactions were conducted under the following conditions: 5 min at 94° C. followed by 30 cycles of 94° C. for 1 min, 54° C. for 1 min, and 72° C. for 1 min; followed by a 5 min final extension at 72° C. Amplified products were analyzed by electrophoresis in a 2% agarose gel followed by ethidium bromide staining.

As a result, FIG. 3 shows that the dT₁₀-ACP1 containing a deoxyinosine group produced almost no products, whereas the dT₁₀-JYC2 lacking a deoxyinosine group produced a plurality of amplified cDNA products. Consistent with our assumption, the results clearly indicate that the deoxyinosine group positioned between the 3′-and 5′-end portions is capable of interrupting the annealing of the 3′-end portion of the dT₁₀-ACP to the template cDNA.

### Example 2

### Identification and Characterization of Differentially Expressed mRNAs During Mouse Embryonic Development Using ACP System

The ACP system of the subject invention has been applied to detect differentially expressed mRNAs during embryonic development. Specifically, the procedure and results using different stages of fetal total RNAs as starting materials are described herein. The primers used in the subject invention are shown in Table 2.

| The cDNA synthesis primer was: | | |
|---|---|---|
| dT₁₀-ACP1 | 5′-GCTTGACTACGATACTGTGCGAIIIIITTTTTTTTTT-3′. | (SEQ ID NO. 30) |
| The following ACPs were used as arbitrary ACPs (ARACPs) for the first | | |
| PCR amplification; | | |
| ACP3 | 5′-GTCTACCAGGCATTCGCTTCATIIIIIGCCATCGACS-3′ | (SEQ ID NO. 3) |
| ACP5 | 5′-GTCTACCAGGCATTCGCTTCATIIIIIAGGCGATGCS-3′ | (SEQ ID NO. 5) |
| ACP8 | 5′-GTCTACCAGGCATTCGCTTCATIIIIICTCCGATGCS-3′ | (SEQ ID NO. 8) |
| ACP10 | 5′-GTCTACCAGGCATTCGCTTCATIIIIIGCCATCGACC-3′ | (SEQ ID NO. 13) |
| ACP13 | 5′-GTCTACCAGGCATTCGCTTCATIIIIIAGGCGATGCG-3′ | (SEQ ID NO. 16) |
| ACP14 | 5′-GTCTACCAGGCATTCGCTTCATIIIIICTCCGATGCC-3′ | (SEQ ID NO. 17) |

The 5′-end portion sequences of the dT₁₀-ACP1 and ARACPs were used as universal primer sequences only for the second PCR amplification. The following primers are the universal primer sequences:

| | | |
|---|---|---|
| JYC2 | 5′-GCTTGACTACGATACTGTGCGA-3′ | (SEQ ID NO. 10) |
| JYC4 | 5′-GTCTACCAGGCATTCGCTTCAT-3′ | (SEQ ID NO. 12) |

For the first PCR amplification, one of the ARACPs (ACP3, ACP5, ACP8, ACP10, ACP13, or ACP14) and the dT₁₀-ACP1 were used as 5′ and 3′ primers, respectively. For the second PCR amplification, JYC4 and JYC2 were used as universal 5′ and 3′ primers, respectively. FIG. 4 shows the amplified cDNA products obtained from different stages of mouse fetal samples using the following sets of primers; the lanes 1-3 of FIG. 4A are with a set of ACP3 and dT₁₀-ACP1; the lanes 1-6 and 7-12 of FIG. 4B are with a set of ACP5 and dT₁₀-ACP1 and a set of ACP8 and dT₁₀-ACP1, respectively. FIG. 5 also shows additional results of the amplified cDNA products using other ACP sets. FIG. 5 shows the amplified products using two sets of the ACP10 and dT₁₀-ACP1(FIG. 5A), and ACP14 and dT₁₀-ACP1 (FIG. 5B), respectively. Many differentially expressed bands in a specific stage were obtained, subcloned into the pGEM-T Easy vector (Promega), and sequenced. Sequence analysis reveals that all of the clones are known genes except two novel genes (Table 1). The expression patterns were confirmed by Northern blot analysis using mouse fetal stage blot (Seegene, Inc., Seoul, Korea). The specific differential display experimental procedure using ACP system is described below.

A. First-Strand cDNA Synthesis

The first-strand cDNAs were prepared under the same conditions as used in the cDNA synthesis of example 1 using the dT₁₀-ACP1 as a cDNA synthesis primer. The resultant cDNAs were purified by a spin column (PCR purification Kit, QIAGEN) to remove primers, dNTP, and the above reagents and cDNAs concentration was determined using the UV spectroscopy at an absorbance of 260 nm. The same amount of cDNAs from each sample was used for comparing their amplification patterns using the ACP system described herein.

B. First PCR cDNA Amplification Using ACP.

The first-strand cDNAs produced from step A are amplified by the following first PCR amplification using one of ARACPs (ACP3, ACP5, ACP8, ACP10, ACP13, or ACP14) and the dT₁₀-ACP1 as 5′ and 3′ primers, respectively. The first PCR amplification was conducted in a 50 μl volume containing 50 ng of the first-strand cDNA, 5 μl of 10×PCR reaction buffer (Promega), 3 μl of 25 mM MgCl₂, 5 μl of dNTP (0.2 mM each dATP, dCTP, dGTP, dTTP), 5 μl of 5′ primer (1 μM), 5 μl of 3′ primer (1 μM), and 0.5 μl of Taq polymerase (5 units/μl). The PCR reactions were as follows: 5 min at 94° C. followed by 20 cycles of 94° C. for 1 min, 50° C. for 1 min, and 72° C. for 1 min; followed by a 5 min final extension at 72° C.

The cycle of the first PCR amplification can be various in accordance with the types of samples. For example, the 20 cycles of the first PCR amplification were used for mouse fetal samples.

C. Second PCR cDNA Amplification Using Universal Primers Derived from the 5′-end Portion Sequences of ACPs.

The amplified cDNA products produced from step B are re-amplified by the following second PCR amplification using two universal primers, JYC4 and JYC2, each corresponding to the 5′-end portion sequences of ARACP and dT₁₀-ACP1, respectively. The second PCR amplification was conducted in a 50 μl volume containing 5 μl of the first amplified cDNA products (50 μl), 5 μl of 10×PCR reaction buffer (Promega), 3 μl of 25 mM MgCl₂, 5 μl of 2 mM dNTP, 1 μl of 5′ primer (10 μM), 1 μl of 3′ primer (10 μM), and 0.5 μl of Taq polymerase (5 units/μl). The PCR reactions were as follows: 5 min at 94° C. followed by 30 cycles of 94° C. for 1 min, 65° C. for 1 min, and 72° C. for 1 min; followed by a 5 min final extension at 72° C.

D. Separation of Amplified PCR Products by Electrophoresis Analysis and Recovery of the Differentially Displayed Bands.

The amplified products were analyzed by electrophoresis in a 2% agarose gel and detected by staining with ethidium bromide. Several major bands differentially expressed during embryonic development (E4.5, E11.5, and E18.5) were selected, excised and extracted from the gels using GENECLEAN II Kit (BIO 101).

E. Re-Amplification of the Recovered Bands.

The bands obtained from step D were re-amplified by the same universal primers and PCR conditions as used in step C.

F. Cloning and Sequencing of the Re-Amplified Fragments.

Each amplified fragment was cloned into the pGEM-T Easy vector (Promega) and sequenced with the ABI PRISM 310 Genetic Analyzer (Perkin Elmer Biosystem) using BigDye Terminator cycle sequencing kit (Perkin Elmer). Computer-assisted sequence analysis was carried out using the BLAST search program (Basic Local Alignment Search Tool).

G. Northern Analysis.

Twenty micrograms of total RNA from fetal tissues were resolved on denaturing 1% agarose gels containing formaldehyde, transferred onto nylon membranes (Hybond-N, Amersham, USA), and hybridized with a ³²P-labeled subcloned PCR product in QuikHyb solution (Stratagene, USA) overnight at 58° C. as previously described (Chun et al., 1999; Hwang et al., 2000). Blots were washed at 65° C. twice for 20 min in 2×SSC, 0.1% SDS, twice for 20 min in 1×SSC, 0.1% SDS, and twice for 20 min in 0.1×SSC, 0.1% SDS. The membranes were exposed to Kodak X-Omat XK-1 film with a Fuji intensifying screen at −80° C.

Differentially expressed clones were isolated and examined for their expression patterns. Consistent with the results of agarose gel analysis, Northern blot analysis showed that the expression patterns of the clones are identical to the original bands on the agarose gels, indicating that all of the clones are true positive products. Thus, the ACP system produces only positive products without any false positives, which means that the ACP system eliminates the problem of false positives. FIG. 6 shows the results of Northern blots for representing six different clones. One of the clones, DEG6, was further examined for its expression during embryonic development. DEG6, which is turned out as a novel gene, shows an interesting expression patterns: after a strong expression appeared at early pregnant stage (E4.5), the expression patterns were gradually reduced, however, its expression was recovered at late development stage (E17.5 and E18.5) (FIG. 7).

TABLE 1
Differentially expressed cDNA fragments cloned by
the ACP system of the present invention

| Nomenclature | Identity | Homology |
|---|---|---|
| DEG 1 | Tropomyosin 2 (beta) | Mouse 92% |
| DEG 2 | Novel | Novel |
| DEG 3 | Hypothetical protein (Tes gene) | Mouse 99% |
| DEG 4 | Protease-6 | Mouse 92% |
| DEG 5 | Novel | Novel |
| DEG 6 | Cytochrome c oxidase, subunit Vb | Mouse 99% |
| DEG 7 | Hydroxylacyl-Coenzyme A dehydrogense | Mouse 98% |
| | (Hadh) | |
| DEG 8 | Troponin T2, cardiac (Tnnt2) | Mouse 94% |
| DEG 9 | RNA binding motif protein, | Mouse 96% |
| | X chromosome | |
| DEG 10 | Peroxiredoxin 6 (Prdx6) | Mouse 89% |
| DEG 11 | 11 days or 13 days embryo cDNA | Mouse 98% |

TABLE 2

| Seq. ID | | |
|---|---|---|
| No. | ID Designation | Sequence information |
| 1 | ACP1 | 5′-GTCTACCAGGCATTCGCTTCATIIIIICAGGAGTGG-3′ |
| 2 | ACP2 | 5′-GTCTACCAGGCATTCGCTTCATIIIIIGGCGACGATS-3′ |
| 3 | ACP3 | 5′-GTCTACCAGGCATTCGCTTCATIIIIIGCCATCGACS-3′ |
| 4 | ACP4 | 5′-GTCTACCAGGCATTCGCTTCATIIIIIAGATGCCCGW-3′ |
| 5 | ACP5 | 5′-GTCTACCAGGCATTCGCTTCATIIIIIAGGCGATGCS-3′ |
| 6 | ACP6 | 5′-GTCTACCAGGCATTCGCTTCATIIIIITCTCCCGGTS-3′ |
| 7 | ACP7 | 5′-GTCTACCAGGCATTCGCTTCATIIIIITTGTGGCGGS-3′ |
| 8 | ACP8 | 5′-GTCTACCAGGCATTCGCTTCATIIIIICTCCGATGCS-3′ |
| 9 | ACP9 | 5′-GTCTACCAGGCATTCGCTTCATIIIIICCTGCGGGTW-3′ |
| 10 | JYC2 | 5′-GCTTGACTACGATACTGTGCGA-3′ |
| 11 | JYC3 | 5′-TCACAGAAGTATGCCAAGCGA-3′ |
| 12 | JYC4 | 5′-GTCTACCAGGCATTCGCTTCAT-3′ |
| 13 | ACP10 | 5′-GTCTACCAGGCATTCGCTTCATIIIIIGCCATCGACC-3′ |
| 14 | ACP11 | 5′-GTCTACCAGGCATTCGCTTCATIIIIIGCCATCGACG-3′ |
| 15 | ACP12 | 5′-GTCTACCAGGCATTCGCTTCATIIIIIAGGCGATGCC-3′ |
| 16 | ACP13 | 5′-GTCTACCAGGCATTCGCTTCATIIIIIAGGCGATGCG-3′ |
| 17 | ACP14 | 5′-GTCTACCAGGCATTCGCTTCATIIIIICTCCGATGCC-3′ |
| 18 | ACP15 | 5′-GTCTACCAGGCATTCGCTTCATIIIIICTCCGATGCG-3′ |
| 19 | CRP2I0 | 5′-GTCTACCAGGCATTCGCTTCATGCCATCGACC-3′ |
| 20 | ACP16 | 5′-GTCTACCAGGCATTCGCCTTCATIIGCCATCGACC-3′ |
| 21 | ACP17 | 5′-GTCTACCAGGCATTCGCTTCATIIIIGCCATCGACC-3′ |
| 22 | ACP18 | 5′-GTCTACCAGGCATTCGCTTCATIIIIIIGCCATCGACC-3′ |
| 23 | ACP19 | 5′-GTCTACCAGGCATTCGCTTCATLIIIIIIIGCCATCGACC-3′ |
| 24 | dT-JYC3 | 5′-TCACAGAAGTATGCCAAGCGACTCGAGTTTTTTTTTTTTTTT-3′ |
| 25 | dT-JYC2 | 5′-GCTTGACTACGATACTGTGCGATTTTTTTTTTTTTTT-3′ |
| 26 | JYC2-T13C | 5′-GCTTGACTACGATACTGTGCGATTTTTTTTTTTTTC-3′ |
| 27 | JYC2-T13G | 5′-GCTTGACTACGATACTGTGCGATTTTTTTTTTTTTG-3′ |
| 28 | JYC2-T13A | 5′-GCTTGACTACGATACTGTGCGATTTTTTTTTTTTTA-3′ |
| 29 | dT₁₀-JYC2 | 5′-GCTTGACTACGATACTGTGCGATTTTTTTTTT-3′ |
| 30 | dT₁₀-ACP1 | 5′-GCTTGACTACGATACTGTGCGAIIIIITTTTTTTTTT-3′ |
| 31 | DEG 2 | GCCATCGACCCGTTTCTCTAGCCCCATCTTCATGTGTTTTA |
| | | ATGAGATGATATTAATTCATTACATTCATGGATAATATGTCCC |
| | | TGAGTACATTCTAATCTAGATTTAACTTCAAAAAAAAAAAAAA |
| | | AAA |
| 32 | DEG 5 | AGGCGATGCGGGCTGTACTCTGGGTGGCTGCCACAGTCTCA |
| | | TGAGAAACCAAGGGCAAAGGACCAAGGAAAAGGGTCTCAGG |
| | | CCCCTAAAGCAGTGGCTTTCAACCATCCTAATGTTGTGACCTT |
| | | TTAATACAGTTCCTCATGTTGTGTGACCCCCCAACCATAAAATG |
| | | ATTTTTGTTTCTACTTCAAAA AAAAAAAAAA AAAAAAAA |

S = G or C
W = A or T
I is deoxyinosine

### REFERENCES

Bauer, D., Muller, H., Reich, J., Ahrenkiel, V., Warthoe, P., Strauss, M. (1993) Identification of differentially expressed mRNA species by an improved display technique (DDRT-PCR). Nucleic Acids Res. 21, 4272-4280.

Bauer, D., Warthoe, P., Rohde, M., Struss, M. (1994) PCR Methods & App: Manual Supplement., pp. S97-S108. Cold Spring Harbor Laboratory, Cold Spring Harbor, N.Y.

Chun, J. Y., Han, Y. J., Ahn, K. Y. (1999) Psx homeobox gene is X-linked and specifically expressed in trophoblast cells o mouse placenta. Dev. Dyn. 216, 257-266

Combates, N., Pardinas, J. R., Parimoo, S., Prouty, S. M., Stenn, K. S. Technique for differential display. U.S. Pat. No. 6,045,998. Date of Patent: Apr. 4, 2000.

Diachenko, L. B., Ledesma, J., Chenchik, A. A., Siebert, P. D. (1996) Combining the technique of RNA fingerprinting and differential display to obtain differentially expressed mRNA. Biochem. Biophys. Res. Commun. 219, 824-828.

Dieffenbach, C. W., Lowe, T. M. J, Dveksler, G. S. (1995) General concepts for PCR primer design. PCR primer: a Laboratory Manual., pp. 133-142, Cold Spring Harbor Laboratory Press, Cold Spring Harbor. N.Y.

Gottschlich, S., Goeoegh, t., Folz, B. J., Lippert, B. M., Werner, J. A. (1997) Optimized differential display and reamplification parameters for silver staining. Res. Commun. Mol. Path. Pharm. 97, 237-240.

Gromova, I., Gromov, P., Celis, J. E. (1999) Identification of true differentially expressed mRNAs in a pair of human bladder transitional cell carcinomas sing an improved differential display procedure. Electrophoresis 20, 241-248.

Hogan, B., Bedding, R., Costantini, F., Lacy, E. (1994) Manipulating the moue embryo: a laboratory manual. Cold Spring Harbor Laboratory Press, Cold Spring Harbor, N.Y.

Hwang, I. T., Lee, Y. H., Moon, B. C., Ahn, K. Y., Lee, S. W., Chun, J. Y. (2000) Identification and characterization of a new member of the placental prolactin-like protein-C (PLP-C) subfamily, PLP-Cβ. Endocrinology 141, 3343-3352.

Ito, T., Kito, K., Adati, N., Mitsui, Y., Hagiwara, H., Sakaki, Y. (1994) Fluorescent differential display: arbitrarily primed RT-PCR fingerprinting on an automated DNA sequencer. FEBS Lett. 351, 231-236.

Jefferies, D., Botman, M. F., Lester, D, Whitehead, C. C., Thorp, B. H. (1998) Cloning differentially regulate genes from chondrocytes using agarose gel differential display. Biochim. Biophys. Acta 1396, 237-241.

Kociok, N., Unfried, K., Eser, P., Krott, R., Schraermeyer, U., Heimann, K. (1998) The nonradioisotopic representation of differentially expressed mRNA by a combination of RNA fingerprinting and differential display. Mol. Biotechnol. 9, 25-33.

Korn, B., Sedlacek, Z., Manca, A., Kioschis, P., Konecki, D., Lehrach, H., Poutska, A. (1992) A strategy for the selection of transcribed sequences in the Xq28 region. Hum. Mol. Genet. 1, 235-242.

Liang, P., Pardee, A. B. (1992) Differential display of eukaryotic messenger RNA by means of the polymerase chain reaction. Science 257, 967-971.

Loakes, D., Brown, D. M. (1994) 5-Nitroindole as an universal base analog. Nucleic Acids Res. 22, 4039-4043.

Luehrsen, K. R., Marr, L. L., Van Der Knaap, E., Cumberledge, S. (1997) Analysis of differential display RT-PCR products using fluorescent primers and genescan software. BioTechniques 22, 168-174.

Matz, M. V., Lukyanov, S. A. (1998) Different strategies of differential display: areas of application. Nucleic Acids Res. 26, 5537-5543.

McPherson, M. J., Moller, S. G. (2000) PCR. BIOS Scientific Publishers, Springer-Verlag New York Berlin Heidelberg, NY.

Mullis, K. B., Erlich, H. A, Arnheim, N., Horn, G. T., Saiki, R. K., Scharf, S. J. Process for amplifying, detecting, and/or cloning nucleic acid sequences. U.S. Pat. No. 4,683,195. Date of Patent: Jul. 28, 1987.

Mullis, K. B. Process for amplifying nucleic acid sequences. U.S. Pat. No. 4,683,202. Date of Patent: Jul. 28, 1987.

Mullis, K. B., Erlich, H. A, Arnheim, N., Horn, G. T., Saiki, R. K., Scharf, S. J. Process for amplifying, detecting, and/or cloning nucleic acid sequences. U.S. Pat. No. 4,800,159. Date of Patent: Jan. 24, 1989.

Nichols, R., Andrews, P. C., Ahang, P., Bergstrom, D. E. (1994) A universal nucleoside for use at ambiguous sites in DNA primers. Nature 369, 492-493.

Ohtsuka, E., Matsuka, S., Ikehara M., Takahashi, Y., Matsubara K. (1985) An alternative approach to deoxyoligonucleotides as hybridization probes by insertion of deoxyinosine at ambiguous codon positions. J. Biol. Chem. 260, 2605-2608.

Rompf, R., Kahl, G. (1997) mRNA differential display in agarose gels. BioTechniques 23, 28-32.

Rosok, O., Odeberg, J., Rode, M., Stokke, T., Funderud, S., Smeland, E. (1996) solid-phase method for differentially display of genes expressed in hematopoietic stem cells. BioTechniques 21, 114-121.

Saiki, R. K., Scharf, S., Faloona, F., Mullis, K. B., Horn, G. T., Erlich, H. A., Arnheim, N. (1985) Enzymatic amplification of beta-globin genomic sequences and restriction site analysis for diagnosis of sickle cell anemia. Science 230, 1350-1354.

Sakanari, J. A., Staunton, C. E., Eakin, A. E., Craik, C. S. (1989) Serine proteases from nematode and protozoan parasites: Isolation of sequence homologs using generic molecular probes. Proc. Natl. Acad. Sci. 86, 4863-4867.

Smith, N. R., Aldersley, M., Li, A., High, A. S., Moynihan, T. P., Markham, A. F., Robinson. P. A. (1997) Automated differential display using a fluorescently labeled universal primer. BioTechniques 23, 274-279.

Sompayrac, L., Jane, S., Burn, T. C., Tene, D. G., Danna, K. J. (1995) Overcoming limitations of the mRNA differential display technique. Nucleic Acids Res. 23, 4738-4739.

Tagle, D. A., Swaroop, M., Lovett, M., Collins, F. S. (1993) Magnetic bead capture of expressed sequences encoded within large genomic segments. Nature 361. 751-753.

Villeponteau, B., Feng, J., Funk, W., Linskens, M. H. K. Method and kit for enhanced differential display. U.S. Pat. No. 5,580,726. Date of Patent: Dec. 3, 1996.

Welsh, J., McClelland, M. (1990) Fingerprinting genomes using PCR with arbitrary primers. Nucleic Acids Res. 18, 7213-7218.

Welsh, J., McClelland, M. (1991) Genomic fingerprinting using arbitrarily primed PCR and a matrix of pairwise combinations of primers. Nucleic Acids Res. 19, 5275-5279.

Williams, J. G. K., Kubelik, A. R., Livak, K. J., Rafalki, J. A., Tingey, S. V. (1990) DNA polymorphisms amplified by arbitrary primers are useful as genetic markers. Nucleic Acids Res. 18, 6531-6535.

### OTHER REFERENCES

Saiki, R. K., Scharf, S., Faloona, F., Mullis, K. B., Horn, G. T., Erlich, H. A., Arnheim, N. (1985) Enzymatic amplification of beta-globin genomic sequences and restriction site analysis for diagnosis of sickle cell anemia. Science 230, 1350-1354.

Mullis, K. B. Process for amplifying nucleic acid sequences. U.S. Pat. No. 4,683,202. Date of Patent: Jul. 28, 1987.

Mullis, K. B., Erlich, H. A, Arnheim, N., Horn, G. T., Saiki, R. K., Scharf, S. J. Process for amplifying, detecting, and/or cloning nucleic acid sequences. U.S. Pat. No. 4,683,195. Date of Patent: Jul. 28, 1987.

Mullis, K. B., Erlich, H. A, Arnheim, N., Horn, G. T., Saiki, R. K., Scharf, S. J. Process for amplifying, detecting, and/or cloning nucleic acid sequences. U.S. Pat. No. 4,800,159. Date of Patent: Jan. 24, 1989.

Ohtsuka, E., Matsuka, S., Ikehara M., Takahashi, Y., Matsubara K. (1985) An alternative approach to deoxyoligonucleotides as hybridization probes by insertion of deoxyinosine at ambiguous codon positions. J. Biol. Chem. 260, 2605-2608.

Nichols, R., Andrews, P. C., Ahang, P., Bergstrom, D. E. (1994) A universal nucleoside for use at ambiguous sites in DNA primers. Nature 369, 492-493.

Loakes, D., Brown, D. M. (1994) 5-Nitroindole as an universal base analog. Nucleic Acids Res. 22, 4039-4043.

Dieffenbach, C. W., Lowe, T. M. J, Dveksler, G. S. (1995) General concepts for PCR primer design. PCR primer: a Laboratory Manual., pp. 133-142, Cold Spring Harbor Laboratory Press, Cold Spring Harbor. NY.

McPherson, M. J., Moller, S. G. (2000) PCR. BIOS Scientific Publishers, Springer-Verlag New York Berlin Heidelberg, NY.

Bauer, D., Warthoe, P., Rohde, M., Struss, M. (1994) PCR Methods & App: Manual Supplement., pp. S97-S108. Cold Spring Harbor Laboratory, Cold Spring Harbor, N.Y.

Liang, P., Pardee, A. B. (1992) Differential display of eukaryotic messenger RNA by means of the polymerase chain reaction. Science 257, 967-971.

Combates, N., Pardinas, J. R., Parimoo, S., Prouty, S. M., Stenn, K. S. Technique for differential display. U.S. Pat. No. 6,045,998. Date of Patent: Apr. 4, 2000.

Rompf, R., Kahl, G. (1997) mRNA differential display in agarose gels. BioTechniques 23, 28-32.

Matz, M. V., Lukyanov, S. A. (1998) Different strategies of differential display: areas of application. Nucleic Acids Res. 26, 5537-5543.

What is claimed is:

1. An annealing control primer comprising a 3′-end portion and a 5′-end portion separated by at least one deoxyinosine residue, universal base or non-discriminatory base analog, wherein the 3′-end portion anneals to a site on a template nucleic acid at a first annealing temperature and the 5′-end portion comprises a universal primer sequence for subsequent amplification of reaction product generated from annealing and extension of the 3′-end portion sequence to the template nucleic acid.

2. The annealing control primer of claim 1, wherein said annealing control primer has a general formula of 5′-dNₓ-dI_{y}-dN_{z}-3′, wherein dNₓ represents the 5′-end portion and contains a pre-selected arbitrary nucleotide sequence; dN_{z} represents the 3′-end portion; dI represents at least one deoxyinosine; x, y, and z each independently represent an integer, wherein x is the number of nucleotides in the 5′-end portion, y is the number of deoxyinosine residues separating the 5′-end portion and 3′-end portion and z is the number of nucleotides in the 3′-end portion.

3. The annealing control primer of claim 2 wherein y is at least 3.

4. The annealing control primer of claim 3, wherein x is an integer of 15 to 40.

5. The annealing control primer of claim 3, wherein y is an integer of 3 to 10.

6. The annealing control primer of claim 3, wherein z is an integer of 6 to 20.

7. The annealing control primer of claim 3, wherein z is less than x.

8. The annealing control primer of claim 3, wherein the sum of x, y, and z is at least 35.

9. The annealing control primer of claim 2 wherein x is an integer of 15 to 40, y is an integer of 3 to 10 and z is an integer of 6 to 20.

10. The annealing control primer of claim 2, wherein y is at least 4.

11. The annealing control primer of claim 2, wherein y is at least 5.

12. The annealing control primer of claim 2, wherein y is at least 6.

13. The annealing control primer of claim 3, wherein dN_{z} is a random nucleotide sequence.

14. The annealing control primer of claim 3, wherein dN_{z} is substantially complementary to a target sequence in the template nucleic acid.

15. The annealing control primer of claim 3, wherein dN_{z} is a degenerate sequence comprising a plurality of combinations of nucleotides encoding a predetermined amino acid sequence.

16. The annealing control primer of claim 1, wherein said annealing control primer has a general formula of 5′-dNₓ-dI_{y}-dN_{z}-3′, wherein dNₓ represents the 5′-end portion and contains a pre-selected arbitrary nucleotide sequence; dN_{z} represents the 3′-end portion; dI represents at least one universal base or non-discriminatory base analog; x, y, and z each independently represent an integer, wherein x is the number of nucleotides in the 5′-end portion, y is the number of universal basees or non-discriminatory base analogs separating the 5′-end portion and 3′-end portion, and z is the number of nucleotides in the 3′-end portion.

17. The annealing control primer of claim 16, wherein said dI is a non-discriminatory base analogue.

18. The annealing control primer of claim 16 wherein dI is a universal base.

19. The annealing control primer of claim 17, wherein said dI is 1-(2′-deoxy-beta-D-ribofuranosyl)-3-nitropyrrole.

20. The annealing control primer of claim 16, wherein said dI is 5-Nitroindole.

21. A kit comprising at least one annealing control primer comprising a 3′-end portion and a 5′-end portion separated by at least one deoxyinosine residue, universal base or non-discriminatory base analog, wherein the 3′-end portion anneals to a site on a template nucleic acid at a first annealing temperature and the 5′-end portion comprises a universal primer sequence for subsequent amplification of reaction product generated from annealing and extension of the 3′-end portion sequence to the template nucleic acid.

22. The kit of claim 21 comprising at least one annealing control primer comprising a 3′-end portion and a 5′-end portion separated by at least three deoxyinosine residues, wherein the 3′-end portion anneals to a site on a template nucleic acid at a first annealing temperature and the 5′-end portion comprises a universal primer sequence for subsequent amplification of reaction product generated from annealing and extension of the 3′-end portion sequence to the template nucleic acid.

23. A process for selectively amplifying a target nucleic acid sequence from a nucleic acid molecule or mixture of nucleic acids, said process comprising carrying out a two-stage polymerase chain reaction (PCR) comprising:
(1) amplifying the target nucleic acid sequence in a first-stage PCR comprising at least one cycle of primer annealing, primer extending and denaturing, by annealing a pair of annealing control primers to the target nucleic acid sequence at a first annealing temperature, wherein the annealing control primers comprise a 3′-end portion and a 5′-end portion separated by at least one deoxyinosine residue, universal base or non-discriminatory base analog, and wherein the 3′-end portion anneals to the target nucleic acid at the first annealing temperature and wherein the 5′-end portion comprises a universal primer sequence; extending the primers to obtain first amplification product; denaturing the first amplification product to obtain denatured amplification product; and
(2) re-amplifying the denatured amplification product in a second-stage PCR comprising at least one cycle of annealing, primer extending and denaturing under high stringency conditions, by annealing the universal primer sequence of the 5′-end portion of the annealing control primers to the universal primer sequences located at the 5′ ends of the denatured amplification product and extending the primers to generate second amplification product.

24. The process according to claim 23, wherein said target nucleic acid is DNA.

25. The process according to claim 23 wherein the target nucleic acid is RNA.

26. The process according to claim 23, wherein the second-stage PCR is repeated at least 10 times.

27. The process according to claim 23, wherein the first annealing temperature is at least 40° C.

28. The process according to claim 23, wherein the annealing in step (2) is carried out at a temperature of at least 50° C.

29. The process according to claim 23, further comprising separating the second amplification product by gel electrophoresis.

30. The process of claim 29 wherein the second amplification product is visualized by ethidium bromide staining the gel.

31. A method for detecting DNA complementary to differentially expressed mRNA in two or more nucleic acid samples comprising:
(a) providing a first sample of nucleic acids representing a first population of mRNA transcripts and a second sample of nucleic acids representing a second population of mRNA transcripts;
(b) separately contacting each of said first nucleic acid sample and said second nucleic acid sample with a first annealing control primer, wherein said first annealing control primer has a hybridizing sequence substantially complementary to the differentially expressed mRNA to hybridize therewith;
(c) reverse transcribing said differentially expressed mRNA to which said first annealing control primer hybridizes to produce a first population of DNA strands that are complementary to said differentially expressed mRNA in said first nucleic acid sample to which said first annealing control primer hybridizes, and a second population of DNA strands that are complementary to said mRNA in said second nucleic acid sample to which said first annealing control primer hybridizes;
(d) purifying and quantifying each of said first and second populations of complementary DNA strands;
(e) contacting each of said first and second populations of DNA strands with a second annealing control primer, wherein said second annealing control primer has a hybridizing sequence substantially complementary to said first and second populations of DNA strands;
(f) extending said second annealing control primer using DNA polymerase to produce a second DNA strand complementary to each of said first and second populations of DNA strands;
(g) amplifying each second DNA strand obtained from step (f) by at least one PCR cycle comprising denaturing, annealing and primer extension to obtain first and second populations of first amplification products;
(h) amplifying the first amplification products generated from step (g) by at least one PCR cycle comprising denaturing, annealing and primer extension to generate first and second populations of second amplification products using two universal primers each comprising a sequence corresponding to each 5′-end portion of said first and second annealing control primers; and
(i) comparing the amount of individual amplification products present in said first and second populations of amplification products.

32. The method according to claim 31, wherein said first nucleic acid sample comprises mRNA expressed in a first cell and said second nucleic acid sample comprises mRNA expressed in a second cell.

33. The method according to claim 31, wherein said first nucleic acid sample comprises mRNA expressed in a cell at a first developmental stage and said second nucleic acid sample comprises mRNA expressed in said cell at a second developmental stage.

34. The method according to claim 31 wherein said first nucleic acid sample comprises mRNA expressed in a tumorigenic cell and said second nucleic acid sample comprises mRNA expressed in a normal cell.

35. The method according to claim 31, wherein said first annealing control primer has a general formula of 5′-dNₓ-dI_{y}-dT_{z}-3′, wherein dNₓ represents the 5′-end portion and contains a pre-selected arbitrary nucleotide sequence; dT_{z} represents the 3′-end portion and dT represents deoxythymidine; dI represents at least one deoxyinosine; x, y, and z each independently represent an integer, wherein x is the number of nucleotides in the 5′-end portion, y is the number of deoxyinosine residue separating the 5′-end portion and 3′-end portion and is at least 3, and z is the number of deoxythymidines in the 3′-end portion.

36. The method according to claim 35, wherein x represents an integer of 15 to 40.

37. The method according to claim 35, wherein y represents an integer of 3 to 10.

38. The method according to claim 35, wherein z represents an integer of 6 to 20.

39. The method according to claim 35, wherein z is less than x.

40. The method according to claim 35, wherein x is at least 22.

41. The method according to claim 35, wherein said y is at least 4.

42. The method according to claim 35, wherein said y is at least 5.

43. The method according to claim 35, wherein said y is at least 6.

44. The method according to claim 31, wherein said first annealing control primer has a general formula of 5′-dNₓ-dI_{y}-dT_{z}-3′, wherein dNₓ represents the 5′-end portion and contains a pre-selected arbitrary nucleotide sequence; dT_{z} represents the 3′-end portion and dT represents deoxythymidine; dI represents at least one universal base or non-discriminatory base analog; x, y, and z each independently represent an integer, wherein x is the number of nucleotides in the 5′-end portion, y is the number of universal bases or non-discriminatory base analogs separating the 5′-end portion and 3′-end portion and is at least 3, and z is the number of nucleotides in the 3′-end portion.

45. The method according to claim 44, wherein said dI is 1-(2′-deoxy-beta-D-ribofuranosyl)-3-nitropyrrole.

46. The method according to claim 44, wherein said dI is 5-Nitroindole.

47. The method according to claim 35, wherein z is at least 10.

48. The method according to claim 35, wherein the sum of x, y, and z is at least 35.

49. The method according to claim 35, wherein said first annealing control primer is SEQ ID NO.30.

50. The method according to claim 31, wherein said second annealing control primer of step (e) has a general formula of 5′-dNₓ-dI_{y}-dN_{z}-3′, wherein dNₓ represents the 5′-end portion and contains a pre-selected arbitrary nucleotide sequence; dN_{y} represents the 3′-end portion; dI represents at least one deoxyinosine; x, y, and z each independently represent an integer, wherein x is the number of nucleotides in the 5′-end portion, y is the number of deoxyinosine residues separating the 5′-end portion and 3′-end portion and z is the number of nucleotides in the 3′-end portion.

51. The method according to claim 50, wherein x represents an integer of 15 to 40.

52. The method according to claim 50, wherein y represents an integer of 3 to 10.

53. The method according to claim 50, wherein z represents an integer of 6 to 20.

54. The method according to claim 50, wherein z is less than x.

55. The method according to claim 50, wherein the sum of x, y, and z is at least 35.

56. The method according to claim 50, wherein x is at least 22.

57. The method according to claim 50, wherein said y is at least 4.

58. The method according to claim 50, wherein said y is at least 5.

59. The method according to claim 50, wherein said y is at least 6.

60. The method according to claim 50, wherein said dN_{z} contains a random nucleotide sequence.

61. The method according to claim 50, wherein said dN_{z} contains at least 10 nucleotides.

62. The method according to claim 50, wherein said second annealing control primer (ACP) is selected from the group consisting of ACP1 (SEQ ID NO. 1), ACP2 (SEQ ID NO. 2), ACP3 (SEQ ID NO. 3), ACP4 (SEQ ID NO. 4), ACP5 (SEQ ID NO. 5), ACP6 (SEQ ID NO. 6), ACP7 (SEQ ID NO. 7), ACP8 (SEQ ID NO. 8), ACP9 (SEQ ID NO. 9), ACP10 (SEQ ID NO. 13), ACP11 (SEQ ID NO. 14), ACP12 (SEQ ID NO. 15), ACP13 (SEQ ID NO. 16), ACP14 (SEQ ID NO. 17), ACP15 (SEQ ID NO. 18), ACP17 (SEQ ID NO. 21), ACP18 (SEQ ID NO. 22), and ACP19 (SEQ ID NO. 23).

63. The method according to claim 31, wherein said said second annealing control primer of step (e) has a general formula of 5′-dNₓ-dI_{y}-dN_{z}-3′, wherein dNₓ represents the 5′-end portion and contains a pre-selected arbitrary nucleotide sequence; dN_{z} represents the 3′-end portion; dI represents at least one universal base or non-discriminatory base analog; x, y, and z each independently represent an integer, wherein x is the number of nucleotides in the 5′-end portion, y is the number of universal bases or non-discriminatory base analogs separating the 5′-end portion and 3′-end portion and z is the number of nucleotides in the 3′-end portion.

64. The method according to claim 63, wherein said dI is 1-(2′-deoxy-beta-D-ribofuranosyl)-3-nitropyrrole.

65. The method according to claim 63, wherein said dI is 5-Nitroindole.

66. The method according to claim 31, wherein the at least one PCR cycle in step (h) is repeated at least 10 times.

67. The method according to claim 31, wherein the annealing in step (g) is carried out at a temperature of between about 45° C. and 55° C.

68. The method according to claim 31, wherein the annealing in step (g) is carried out at a temperature of at least 50° C.

69. The method according to claim 31, wherein the annealing in step (h) is carried out at a temperature of about between 55° C. and 70° C.

70. The method according to claim 31, wherein the annealing in step (h) is carried out at a temperature of at least 65° C.

71. The method according to claim 31, wherein said universal primers used in step (h) are JYC2 (SEQ ID NO. 10) and JYC4 (SEQ ID NO. 12).

72. A kit comprising the first and second annealing control primers of claim 31.

73. A kit comprising the universal primers of claim 31.

74. The method according to claim 31, wherein the comparing of step (i) comprises resolving each of said first and second populations of amplification products by gel electrophoresis through an ethidium bromide-stained agarose gel and comparing the presence or level of bands of a particular size.

75. The method according to claim 31, wherein the comparing of step (i) comprises resolving each of said first and second populations of amplification products by gel electrophoresis through a denaturing polyacrylamide gel and comparing the presence or level of bands of a particular size.

76. The method according to claim 31, wherein the nucleotide sequence of each of said first and second annealing control primers contains a restriction endonuclease recognition site.

77. The method of claim 31 further comprising isolating the second amplification product.

78. The method of claim 77 further comprising cloning the isolated second amplification product into a vector.

79. The method according to claim 31, wherein at least one of said first and second annealing control primers contains a plurality of deoxyoligonucleotides.

80. A kit comprising the first and second annealing control primers of claim 31.


SEQUENCE LISTING (32 sequences)

SEQ ID NO: 1 - DNA, 37 bp, Artificial Sequence
  Description of Artificial Sequence  primer
gtctaccaggcattcgcttcatnnnnngcaggagtgg

SEQ ID NO: 2 - DNA, 37 bp, Artificial Sequence
  Description of Artificial Sequence primer
gtctaccaggcattcgcttcatnnnnnggcgacgatn

SEQ ID NO: 3 - DNA, 37 bp, Artificial Sequence
  Description of Artificial Sequence primer
gtctaccaggcattcgcttcatnnnnngccatcgacn

SEQ ID NO: 4 - DNA, 37 bp, Artificial Sequence
  Description of Artificial Sequence primer
gtctaccaggcattcgcttcatnnnnnagatgcccgn

SEQ ID NO: 5 - DNA, 37 bp, Artificial Sequence
  Description of Artificial Sequence  primer
gtctaccaggcattcgcttcatnnnnnaggcgatgcn

SEQ ID NO: 6 - DNA, 37 bp, Artificial Sequence
  Description of Artificial Sequence primer
gtctaccaggcattcgcttcatnnnnntctcccggtn

SEQ ID NO: 7 - DNA, 37 bp, Artificial Sequence
  Description of Artificial Sequence primer
gtctaccaggcattcgcttcatnnnnnttgtggcggn

SEQ ID NO: 8 - DNA, 37 bp, Artificial Sequence
  Description of Artificial Sequence primer
gtctaccaggcattcgcttcatnnnnnctccgatgcn

SEQ ID NO: 9 - DNA, 37 bp, Artificial Sequence
  Description of Artificial Sequence primer
gtctaccaggcattcgcttcatnnnnncctgcgggtn

SEQ ID NO: 10 - DNA, 22 bp, Artificial Sequence
  Description of Artificial Sequence primer
gcttgactacgatactgtgcga

SEQ ID NO: 11 - DNA, 20 bp, Artificial Sequence
  Description of Artificial Sequence primer
tcacagaagtatgccagcga

SEQ ID NO: 12 - DNA, 22 bp, Artificial Sequence
  Description of Artificial Sequence primer
gtctaccaggcattcgcttcat

SEQ ID NO: 13 - DNA, 37 bp, Artificial Sequence
  modified primer, wherein ”n“ in positions 23-27 is deoxyinosine (”dI“)
gtctaccaggcattcgcttcatnnnnngccatcgacc

SEQ ID NO: 14 - DNA, 37 bp, Artificial Sequence
  Description of Artificial Sequence primer
gtctaccaggcattcgcttcatnnnnngccatcgacg

SEQ ID NO: 15 - DNA, 37 bp, Artificial Sequence
  Description of Artificial Sequence primer
gtctaccaggcattcgcttcatnnnnnaggcgatgcc

SEQ ID NO: 16 - DNA, 37 bp, Artificial Sequence
  Description of Artificial Sequence primer
gtctaccaggcattcgcttcatnnnnnaggcgatgcg

SEQ ID NO: 17 - DNA, 37 bp, Artificial Sequence
  Description of Artificial Sequence primer
gtctaccaggcattcgcttcatnnnnnctccgatgcc

SEQ ID NO: 18 - DNA, 37 bp, Artificial Sequence
  Description of Artificial Sequence primer
gtctaccaggcattcgcttcatnnnnnctccgatgcg

SEQ ID NO: 19 - DNA, 32 bp, Artificial Sequence
  Description of Artificial Sequence primer
gtctaccaggcattcgcttcatgccatcgacc

SEQ ID NO: 20 - DNA, 34 bp, Artificial Sequence
  Description of Artificial Sequence primer
gtctaccaggcattcgcttcatnngccatcgacc

SEQ ID NO: 21 - DNA, 36 bp, Artificial Sequence
  Description of Artificial Sequence primer
gtctaccaggcattcgcttcatnnnngccatcgacc

SEQ ID NO: 22 - DNA, 38 bp, Artificial Sequence
  Description of Artificial Sequence primer
gtctaccaggcattcgcttcatnnnnnngccatcgacc

SEQ ID NO: 23 - DNA, 40 bp, Artificial Sequence
  Description of Artificial Sequence primer
gtctaccaggcattcgcttcatnnnnnnnngccatcgacc

SEQ ID NO: 24 - DNA, 42 bp, Artificial Sequence
  Description of Artificial Sequence primer
tcacagaagtatgccaagcgactcgagttttttttttttttt

SEQ ID NO: 25 - DNA, 37 bp, Artificial Sequence
  Description of Artificial Sequence primer
gcttgactacgatactgtgcgattttttttttttttt

SEQ ID NO: 26 - DNA, 36 bp, Artificial Sequence
  Description of Artificial Sequence primer
gcttgactacgatactgtgcgatttttttttttttc

SEQ ID NO: 27 - DNA, 36 bp, Artificial Sequence
  Description of Artificial Sequence  primer
gcttgactacgatactgtgcgatttttttttttttg

SEQ ID NO: 28 - DNA, 36 bp, Artificial Sequence
  Description of Artificial Sequence  primer
gcttgactacgatactgtgcgattttttttttttta

SEQ ID NO: 29 - DNA, 32 bp, Artificial Sequence
  Description of Artificial Sequence primer
gcttgactacgatactgtgcgatttttttttt

SEQ ID NO: 30 - DNA, 37 bp, Artificial Sequence
  Description of Artificial Sequence primer
gcttgactacgatactgtgcgannnnntttttttttt

SEQ ID NO: 31 - DNA, 131 bp, mouse
gccatcgacccgtttctctagccccatcttcatgtgttttaatgagatgatattaattcattacattcatggataatatgtaccctgagtacattctaatctagatttaacttcaaaaaaaaaaaaaaaaa

SEQ ID NO: 32 - DNA, 208 bp, mouse
aggcgatgcgggctgtactctgggtggctgccacagtctcatgagaaaccaagggcaaaggaccaaggaaaagggtctcaggcccctaaagcagtggctttcaaccatcctaatgttgtgaccttttaatacagttcctcatgttgtgtgaccccccaaccataaaatgatttttgtttctacttcaaaaaaaaaaaaaaaaaaaaaa